(19) United States
(12) Patent Application Publication
(10) Pub. No.: US 2025/0177298 A1
(43) Pub. Date: Jun. 5, 2025

(54) EMULSIONS AND A METHOD FOR THEIR PREPARATION

(71) Applicant: XAMPLA LIMITED, Cambridge, Cambridgeshire (GB)

(72) Inventors: James Ward TAYLOR, Cambridge, Cambridgeshire (GB); Marc RODRIGUEZ GARCIA, Cambridge, Cambridgeshire (GB); Ayaka KAMADA, Cambridge, Cambridgeshire (GB); Polly Helena Ruth KEEN, Cambridge, Cambridgeshire (GB)

(21) Appl. No.: 18/844,883

(22) PCT Filed: Mar. 8, 2023
(86) PCT No.: PCT/EP2023/055934

§ 371 Date: Sep. 6, 2024

(30) Foreign Application Priority Data
Mar. 8, 2022 (LU) 501621

(51) Int. Cl. A61K 9/107; A61K 8/06; A61K 8/64; A61K 31/07; A61K 31/197; A61K 31/375; A61K 31/4188; A61K 31/4415; A61K 31/455; A61K 31/51; A61K 31/525; A61K 31/593; A61K 31/714; A61K 47/42; A61Q 19/00

(52) U.S. Cl. CPC A61K 9/107; A61K 8/06; A61K 8/645; A61K 31/07; A61K 31/197; A61K 31/375; A61K 31/4188; A61K 31/4415; A61K 31/455; A61K 31/51; A61K 31/525; A61K 31/593; A61K 31/714; A61K 47/42; A61Q 19/00; A61K 2800/10

(57) ABSTRACT

The present invention relates to a method for preparing an emulsion using a plant-based protein hydrogel slurry. The present invention also relates to the emulsions per se and to compositions comprising the emulsions. The present invention also relates to the use of a plant-based protein hydrogel slurry as an emulsifier.

### FIELD OF THE INVENTION

The present invention relates to a method for preparing an emulsion using a plant-based protein hydrogel slurry. The present invention also relates to the emulsions per se and to compositions comprising the emulsions. The present invention also relates to the use of a plant-based protein hydrogel slurry as an emulsifier.

### BACKGROUND

Numerous consumer product applications require a poorly water-soluble active ingredient to be solubilised in water, e.g. in cosmetic formulations such as skin cream. An amphiphilic molecule can be used as an emulsifier to create an oil-in-water emulsion that solubilises the active, and provides some stability for the formulation (i.e. so that the consumer product does not separate out/degrade quickly once made). Conventional amphiphilic emulsifiers are fundamentally thermodynamically unstable and do not provide sufficient emulsion stability over very prolonged periods of time. Fossil fuel-derived materials are also commonly used as emulsifiers, however, there is an increasing desire to employ more sustainable materials. Unfortunately, many materials of natural origin that could be used for this purpose are highly processed, which brings about a new set of problems. For example, many such emulsifiers have foaming properties that are not suited to non-cleansing products, or have poor feel aesthetics meaning that they are sticky when applied topically.

More recently, plant proteins have been investigated for their emulsifying properties. However, conventional plant protein emulsifiers are relatively poor emulsifiers requiring high levels to be present in a formulation to successfully solubilise poorly water-soluble materials. Conventional plant protein emulsifiers tend to also have high levels of soluble proteins present, which are more likely to result in allergic reactions. Thus, the high level of potentially allergenic soluble plant proteins in such formulations limits the use of these materials. For example, such materials cannot be used in a formulation intended for topical application or in an edible formulation, as the risk of an allergic reaction would be too high.

Accordingly, there exists a need to prepare novel emulsifiers that are both sustainable and have a low or negligible allergenicity risk, and which are able to form emulsions having long-term stability.

### SUMMARY OF THE INVENTION

Viewed from a first aspect, the present invention provides a method for the preparation of an emulsion, the method comprising:
(a) forming a solution comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s);
(b) inducing the protein in the solution to undergo a sol-gel transition to form a plant-based protein hydrogel;
(c) subjecting the plant-based protein hydrogel to a first shear step to form a first plant-based protein hydrogel slurry;
(d) subjecting the first plant-based protein hydrogel slurry to a solvent reduction step to form a washed plant-based protein hydrogel slurry;
(e) subjecting the washed plant-based protein hydrogel slurry to a second shear step to form a second plant-based protein hydrogel slurry; and
(f) dispersing a lipophilic phase in the second plant-based protein hydrogel slurry to form the emulsion,
wherein said emulsion has a protein solids content of less than 1 wt % based upon the total weight of the emulsion.

Viewed from a further aspect, the present invention provides an emulsion obtained by or obtainable by the method as hereinbefore described.

Viewed from a further aspect, the present invention provides an emulsion comprising a lipophilic phase dispersed in a plant-based protein hydrogel slurry comprising a plant-based protein(s), wherein said emulsion has a protein solids content of less than 1 wt % based upon the total weight of the emulsion.

Viewed from a further aspect, the present invention provides a composition comprising an emulsion prepared according to the method as hereinbefore described.

Viewed from a further aspect, the present invention provides the use of a plant-based protein hydrogel slurry as an emulsifier in a composition.

### Definitions

As used herein, the term “lower shear step” may refer to a process step in which low levels of mechanical energy are applied to a material, preferably by a cutting action, to cause it to break or fragment primarily into large discrete fragments. “Lower shear” does not typically include any milling step that shatters or fragments a material by high-speed impact, for example impacts at a differential velocity of greater than 2 ms⁻¹. Nor does it typically include milling processes based on cavitation. In a particular embodiment, during the lower shear step, a hydrogel is fragmented to give fragments such that at least 80% by weight of the hydrogel fragments have a maximum dimension, as determined by optical microscopy, of between 1 mm and 100 mm.

As used herein, the term “higher shear step” may refer to a process step which applies energy to reduce the hydrogel to small fragments, such as to form e.g. a colloidal dispersion. In a particular embodiment, during the higher shear step, a hydrogel may be fragmented to give fragments having a de particle size as determined by laser diffraction of less than 100 microns.

For the avoidance of doubt, a higher shear step subjects the hydrogel to higher levels of shear than the lower shear step. In the instance the method involves both a lower shear step and a higher shear step, the higher shear step must happen after the lower shear step (i.e. they are discrete steps occurring in this particular order).

As used herein, the term “sol-gel transition temperature” refers to the temperature at which a plant-based protein transforms from a liquid state into a hydrogel state. Thus, at temperatures above the sol-gel transition temperature, the plant-based protein will be in a liquid state, and at temperatures below the sol-gel transition temperature the plant-based protein will be in a hydrogel state.

As used herein, the term “fragrance” (used interchangeably with the term “perfume”) refers to the component of a formulation that is capable of imparting or modifying the odour of a product, such as a body lotion or a hair conditioner or a substrate such as skin or hair. A fragrance is typically used to impart an overall pleasant odour or odour profile to a product either to provide a pleasurable experience. such as a Fine Fragrance, or to provide sensory cues as to the product's benefit and function, such as a calming effect for a lavender sleep aid, the idea of cleanliness for a laundry product, or to mask an unpleasant odour, such as in an insect repellent product. A “fragrance” may be composed of one or more components that can be a single chemical entity, referred to herein as a “fragrance material” (used interchangeably with the term “perfume material”), or a mixture of different “fragrance materials”. Fragrance materials can be created by either synthetic processes or extracted from nature, particularly plants, to obtain naturally occurring plant essential oils and plant extracts such as orange oil, Fragrance materials created by synthetic processes can be either new-to-the-world chemicals or nature-identical fragrance materials. Synthetic and naturally derived fragrance materials can then be blended into fragrances by skilled perfumers, also called noses, for use in consumer products. Fragrance materials can be obtained from specialist fragrance suppliers, known as fragrance houses, as individual chemicals, natural blends or as proprietary specialty blends where the full composition is not disclosed. The individual fragrance materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994) and “Perfume and Flavour Materials of Natural Origin”, S. Arctander. Ed., Elizabeth, N.J., 1960. It will be understood that for the purposes of this invention, a “fragrance material” includes a pro-fragrance such as an acetal pro-fragrance, ketal pro-fragrance, ester pro-fragrance, hydrolysable inorganic-organic pro-fragrance, and combinations thereof. The fragrance materials may be released from the pro-fragrances in a number of ways, for example, by hydrolysis, or by a shift in an equilibrium reaction, or by a pH-change, or by enzymatic release, or by UV-radiation.

A fragrance material can be described in terms of its odour strength, detection threshold, odour saturation and its character. In fragrance emulsions it is preferable to use fragrance materials with a low odour detection threshold and a high strength so as to maximise the noticeability of even small levels of fragrance encapsulated and released.

In order to impart an odour, a fragrance material must be volatile, even if only to a small degree, as it is necessary for the molecule to be airborne to enter the nose, where it gets attached to specific neuroreceptors and elicits a signal within the olfactory system. Fragrance materials can be classified according to their volatility. Preferably fragrance materials are liquid at 20° C., and atmospheric pressure but occasionally they may be solid and can be blended with other liquid fragrance materials or solvents. Typically the fragrance industry refers to the volatility and substantivity by loosely categorising materials into one of 3 categories: base notes for the least volatile and most substantive, heart notes for those of moderate volatility and substantivity and top notes for the most volatile and least substantive. This is based on the odour perception of the materials and is quite subjective. One way of objectively classifying the volatility of fragrance materials is according to their vapour pressure.

As used herein, the term “vapour pressure” means the partial pressure in air at a defined temperature (e.g. 25° C.) and standard atmospheric pressure (760 mmHg) for a given chemical species. It defines a chemical species' affinity for the gas phase rather than the liquid or solid state. The higher the vapour pressure the greater the proportion of the material that will, at equilibrium, be in a closed headspace. It is also related to the rate of evaporation of a fragrance material which is defined in an open environment where the material is leaving the system. The vapour pressure can be readily determined according to the reference program ACD/Percepta Desktop Software, Version 14.0 (Build: Aug. 26, 2021), Advanced Chemistry Development. Inc (ACD/Labs), Toronto. Canada, www.acdlabs.com.

A physical parameter that is relevant to the encapsulation of a fragrance material is its hydrophobicity, which may be defined in terms of its partition coefficient P. As used herein, the term “partition coefficient” refers to the ratio between the equilibrium concentration of that substance in n-octanol and in water, and is a measure of the differential solubility of said substance between these two solvents. As used herein, the term “log P” refers to the logarithm to the base 10 of the partition coefficient P. The log P can be readily determined according to the reference program ACD/Percepta Desktop Software, Version 14.0 (Build: Aug. 26, 2021), Advanced Chemistry Development, Inc (ACD/Labs), Toronto, Canada, www.acdlabs.com Log P values are predicted from the SMILE string of fragrance materials molecules. Three different types of log P values can be selected from the software. The log P Classic is based on an algorithm which takes into account a database of experimental log P values while using the principal of isolating carbons. The log P GALAS is based on an algorithm taking into account a database of a training set of compounds as well as adjusting the values with data from structurally close compounds. The consensus log P is a model based on the previous two algorithms, which can be expressed as: consensus log P=a×log P Classic+b×log P GALAS, where a and b are coefficients of the model. The latter value, consensus log P, is the log P value 25 indicated herein.

Another aspect that is relevant to the encapsulation of a fragrance material is its Hansen Solubility Parameters (HSPs). The term HSP refers to a solubility parameter approach proposed by Charles Hansen first used to predict polymer solubility in a given solvent as described in. The Three Dimensional Solubility Parameter and Solvent Diffusion Coefficient, by Charles Hansen, Danish Technical Press (Copenhagen, 1967). This approach has since been reapplied to many other molecules. The fragrance material (or flavour material or solvent) and its interactions with its environment are defined by 3 forces: atomic dispersion forces, molecular permanent dipole forces, and molecular hydrogen bonding forces. Materials with similar HSP parameters are more likely to be miscible. These forces can be quantified by 3 values: δD, the Hansen dispersion value which relates to the Van der Waals interactions (intermolecular forces); δP, the Hansen polarity value which relates to the dipole moment (electrical charges); and δH, the Hansen Hydrogen-bonding (“h-bonding”) value. The solubility parameter 6 (MPa^{1/2}) is defined as δ²=δ_{P}²+δ_{H}²=E/V, where E is the cohesive energy of a solvent and V is the molar volume. The HSP values for a given material can be obtained from the HSPiP (Hansen Solubility Parameters in Practice) software available from www.hansen-solubility.com through two main different ways. Those values can either be retrieved from the Master Dataset, which comprises over 20,000 materials, by searching by name or CAS number: or be predicted by entering the SMILE string of a given molecule in the DIY section of the software, using the Y-MB (Yamamoto-Molecular Breaking) method. Furthermore, the determination of the HSP sphere relative to a given fragrance material is a good way to predict the solubility preferences within a blend of fragrance materials. The radius Ro of the HSP sphere is defined as Ro=Ra/RED, where Ra is the HSP distance between two molecules (1 and 2) expressed by: Ra²=4 (δ_{D1}−δ_{D2})²+(δ_{P1}-δ_{P2})²+(δ_{H1}−δ_{H2})², and RED is the Relative Energy Difference. This RED value can also be extracted from or predicted through the HSPiP software, and a good solvent for a given material should exhibit a RED value lower or equal to 1, whereas a solvent displaying a RED value greater than 1 should be considered as a bad solvent for the given material.

A fragrance material may be selected from an alcohol, an aldehyde, a ketone, an ester, an ether, an acetate, an alkene, a nitrile, a nitrogenous heterocyclic compound, a sulfurous heterocyclic compound, and a Schiff base.

Preferred aldehyde fragrance materials include, without limitation, alpha-amylcinnamaldehyde, anisic aldehyde, decyl aldehyde, lauric aldehyde, methyl n-nonyl acetaldehyde, methyl octyl acetaldehyde, nonylaldehyde, benzenecarboxaldehyde, neral, geranial, 1,1-diethoxy-3,7-dimethylocta-2,6-diene, 4-isopropylbenzaldehyde, 2,4-dimethyl-3-cyclohexene-1-carboxaldehyde, alpha-methyl-p-isopropyldihydrocinnamaldehyde, 3-(3-isopropylphenyl) butanal, alpha-hexylcinnamaldehyde, 7-hydroxy-3,7-dimethyloctan-1-al. 2,4-dimethyl-3-cyclohexene-1-carboxaldehyde, octyl aldehyde, phenylacetaldehyde, 2,4-dimethyl-3-cyclohexene-1-carboxaldehyde, hexanal. 3,7-dimethyloctanal. 6,6-dimethylbicyclo[3.1.1]hept-2-ene-2-butanal, nonanal, octanal, 2-nonenal undecenal, 2-methyl-4-(2,6,6-trimethyl-1-cyclohexenyl-1)-2-butenal, 2,6-dimethyloctanal, 3-(p-isopropylphenyl) propionaldehyde, 3-phenyl-4-pentenal citronellal, o/p-ethyl-alpha, alpha, 9-decenal, dimethyldihydrocinnamaldehyde, p-isobutyl-alphamethylydrocinnamaldehyde, cis-4-decen-1-al, 2,5-dimethyl-2-ethenyl-4-hexenal, trans-2-methyl-2-butenal. 3, methylnonanal, alpha-sinensal, 3-phenylbutanal. 2,2-dimethyl-3-phenylpropionaldehyde, m-tertbutyl-alpha-methyldihydrocinnamic aldehyde, geranyl oxyacetaldehyde, trans-4-decen-1-al, methoxycitronellal, and mixtures thereof.

Preferred ester fragrance materials include, without limitation, allyl cyclohexane-propionate, allyl heptanoate, allyl amyl glycolate, allyl caproate, amyl acetate (n-pentyl acetate), amyl propionate, benzyl acetate, benzyl propionate, benzyl salicylate, cis-3-hexenylacetate, citronellyl acetate, citronellyl propionate, cyclohexyl salicylate, dihydro isojasmonate, dimethyl benzyl carbinyl acetate, ethyl acetate, ethyl acetoacetate, ethyl butyrate, ethyl-2-methyl butryrate, ethyl-2-methyl pentanoate, fenchyl acetate (1,3,3-trimethyl-2-norbornanyl acetate), tricyclodecenyl acetate, tricyclodecenyl propionate, geranyl acetate, cis-3-hexenyl isobutyrate, hexyl acetate, cis-3-hexenyl salicylate, n-hexyl salicylate, isobornyl acetate, linalyl acetate, para-tertiary-butyl cyclohexyl acetate, (−)-L-menthyl acetate, ortho-tertiary-butylcyclohexyl acetate, methyl benzoate, methyl dihydro isojasmonate, alpha-methylbenzyl acetate, methyl salicylate, 2-phenylethyl acetate, prenyl acetate, cedryl acetate, cyclabute, phenethyl phenylacetate, terpinyl formate, citronellyl anthranilate, ethyl tricyclo[5.2.1.0-2,6]decane-2-carboxylate, n-hexyl ethyl acetoacetate, 2-tertbutyl-4-methyl cyclohexyl acetate, formic acid, 3,5,5-trimethylhexyl ester, phenethyl crotonate, cyclogeranyl acetate, geranyl crotonate, ethyl geranate, geranyl isobutyrate, 3,7-dimethyl-ethyl 2-nonynoate-2,6-octadienoic acid methyl ester, citronellyl valerate. 2-hexenylcyclopentanone, cyclohexyl anthranilate, L-citronellyl tiglate, butyl tiglate, pentyl tiglate, geranyl caprylate. 9-decenyl acetate, 2-isopropyl-5-methylhexyl-1 butyrate, n-pentyl benzoate. 2-methylbutyl benzoate (and mixtures thereof with pentyl benzoate), dimethyl benzyl carbinyl propionate, dimethyl benzyl carbinyl acetate, trans-2-hexenyl salicylate, dimethyl benzyl carbinyl isobutyrate. 3,7-dimethyloctyl formate, rhodinyl formate, rhodinyl isovalerate, rhodinyl acetate, rhodinyl butyrate, rhodinyl propionate, cyclohexylethyl acetate, neryl butyrate, tetrahydrogeranyl butyrate, myrcenyl acetate, 2,5-dimethyl-2-ethenylhex-4-enoic acid, methyl ester, 2,4-dimethylcyclohexane-1-methyl acetate, ocimenyl acetate, linalyl isobutyrate, 6-methyl-5-heptenyl-1 acetate, 4-methyl-2-pentyl acetate, n-pentyl 2-methylbutyrate, propyl acetate, isopropenyl acetate, isopropyl acetate. 1-methylcyclohex-3-ene-carboxylic acid, methyl ester, propyl tiglate, propyl/isobutyl cyclopent-3-enyl-1-acetate (alphavinyl), butyl 2-furoate, ethyl 2-pentenoate, (E)-methyl 3-pentencate, 3-methoxy-3-methylbutyl acetate, n-pentyl crotonate, n-pentyl isobutyrate, propyl formate, furfuryl butyrate, methyl angelate, methyl pivalate, prenyl caproate, furfuryl propionate, diethyl malate, isopropyl 2-methylbutyrate, dimethyl malonate, bornyl formate, styralyl acetate, 1-(2-furyl)-1-propanone, I-citronellyl acetate, 3,7-dimethyl-1,6-nonadien-3-yl acetate, neryl crotonate, dihydromyrcenyl acetate, tetrahydromyrcenyl acetate, lavandulyl acetate, 4-cyclooctenyl isobutyrate, cyclopentyl isobutyrate, 3-methyl-3-butenyl acetate, allyl acetate, geranyl formate, cis-3-hexenyl caproate, and mixtures thereof.

Preferred alcohol fragrance materials include, without limitation, benzyl alcohol, beta-gamma-hexenol (2-hexen-1-ol), cedrol, citronellol, cinnamic alcohol, p-cresol, cumic alcohol, dihydromyrcenol, 3,7-dimethyl-1-octanol, dimethyl benzyl carbinol, eucalyptol, eugenol, fenchyl alcohol, geraniol, hydratopic alcohol, isononyl alcohol (3,5,5-trimethyl-1-hexanol), linalool, methyl chavicol (estragole), methyl eugenol (eugenyl methyl ether), nerol, 2-octanol, patchouli alcohol, phenyl hexanol (3-methyl-5-phenyl-1-pentanol), phenethyl alcohol, alpha-terpineol, tetrahydrolinalool, tetrahydromyrcenol, 4-methyl-3-decen-5-ol, 1-3,7-dimethyloctane-1-ol, 2-(furfuryl-2)-heptanol. 6,8-dimethyl-2-nonanol, ethyl norbornyl cyclohexanol, beta-methyl cyclohexane ethanol, 3,7-dimethyl-(2),6-octen (adien)-1-ol, trans-2-undecen-1-ol. 2-ethyl-2-prenyl-3-hexenol, isobutyl benzyl carbinol, dimethyl benzyl carbinol, ocimenol. 3,7-dimethyl-1,6-nonadien-3-ol (cis & trans), tetrahydromyrcenol, alpha-terpineol. 9-decenol-1, 2-(2-hexeny)-cyclopentanol, 2,6-dimethyl-2-heptanol, 3-methyl-1-octen-3-ol, 2,6-dimethyl-5-hepten-2-ol, 3,7,9-trimethyl-1,6-decadien-3-ol. 3,7-dimethyl-6-nonen-1-ol, 3,7-dimethyl-1-octyn-3-ol, 2,6-dimethyl-1,5,7-octatrienol-3, dihydromyrcenol, 2.6,-trimethyl-5,9-undecadienol. 2,5-dimethyl-2-propylhex-4-enol-1. (Z)-3-hexenol, o,m,p-methyl-phenylethanol, 2-methyl-5-phenyl-1-pentanol, 3-methylphenethyl alcohol, para-methyl dimethyl benzyl carbinol, methyl benzyl carbinol, p-methylphenylethanol. 3,7-dimethyl-2-octen-1-ol, 2-methyl-6-methylene-7-octen-4-ol, and mixtures thereof.

Preferred ketone fragrance materials include, without limitation, oxacycloheptadec-10-en-2-one, benzylacetone, benzophenone, L-carvone, cis-jasmone, 4-(2,8,6-trimethyl-3-cyclohexen-1-yl)-but-3-en-4-one, ethyl amyl ketone, alpha-ionone, ionone beta, ethanone, octahydro-2,3,8,8-tetramethyl-2-acetonaphthalene, alpha-irone, 1-(5,5-dimethyl-1-cyclo-hexen-1-yl)-4-penten-1-one, 3-nonanone, ethyl hexyl ketone, menthone, 4-methyl-acetophenone, gamma-methyl ionone, methyl pentyl ketone, methyl heptenone (6-methyl-5-hepten-2-one), methyl heptyl ketone, methyl hexyl ketone, delta muscenone, 2-octanone, 2-pentyl-3-methyl-2-cyclopenten-1-one. 2-heptylcyclopentanone, alpha-methylionone, 3-methyl-2-(trans-2-pentenyl)-cyclopentenone, octenyl cyclopentanone, n-amylcyclopentenone, 6-hydroxy-3,7-dimethyloctanoic acid lactone, 2-hydroxy-2-cyclohexen-1-one, 3-methyl-4-phenyl-3-buten-2-one, 2-pentyl-2,5,5-trimethylcyclopentanone, 2-cyclopentylcyclopentanol-1, 5-methylhexan-2-one, gamma-dodecalactone, delta-dodecalactone, gamma-nonalactone, delta-nonalactone, gamma-octalactone, delta-undecalactone, gamma-undecalactone, alpha damascone, beta damascone, gamma damascone, delta damascone and mixtures thereof.

Preferred ether fragrance materials include, without limitation, diphenyl oxide, p-cresyl methyl ether, 4,6,6,7,8,8-hexamethyl-1,3,4,6,7,8-hexahydro-cyclopenta (G)-2-benzopyran, beta-naphthyl methyl ether, methyl isobutenyl tetrahydropyran, 5-acetyl-1,1,2,3,3,6-hexamethylindan (phantolide): 7-acetyl-1,1,3,4,4,6-hexamethyltetralin (tonalid), 2-phenylethyl-3-methylbut-2-enyl ether, ethyl geranyl ether, phenylethyl isopropyl ether, and mixtures thereof.

Preferred alkene fragrance materials include, without limitation, allo-ocimene, camphene, beta-caryophyllene, cadinene, diphenylmethane, d-limonene, lymolene, beta-myrcene, para-cymene. 2-alpha-pinene, beta-pinene, alpha-terpinene, gamma-terpinene, terpineolene, 7-methyl-3-methylene-1,6-octadiene, and mixtures thereof.

Preferred nitrile fragrance materials include, without limitation, 3,7-dimethyl-6-octenenitnie, 3,7-dimethyl-2 (3), 6-nonadienenitrile, (2E,6Z)-2,6-nonadienenitrile, n-dodecane nitrile, and mixtures thereof.

Preferred Schiff base fragrance materials include, without limitation, citronellyl nitrile, nonanal/methyl anthranilate, N-octylidene-anthranilic acid methyl ester, hydroxycitronellal/methyl anthranilate, cyclamen aldehyde/methyl anthranilate, methoxyphenylpropanal/methyl anthranilate, ethyl p-aminobenzoate/hydroxycitronellal, citral/methyl anthranilate. 2,4-dimethylcyclohex-3-enecarbaldehyde methyl anthranilate, hydroxycitronellal-indole, and mixtures thereof.

As used herein, the term “flavour” refers to the component of a formulation that is capable of imparting or modifying the taste and smell of a product, such as a toothpaste or foodstuff. A flavour is typically used to impart an overall pleasant taste and smell, or a taste and smell profile, to a product either to simply provide a pleasurable experience, such as in a foodstuff, or to mask an unpleasant taste or smell, such as in a medicine. A flavour or flavour material can be described in terms of its aroma strength, detection threshold and quality. A “flavour” may be composed of one or more components that can be a single chemical entity, referred to herein as a “flavour material”, or a mixture of different “flavour materials”. Flavour materials can be created by either synthetic processes or extracted from nature, particularly from plants, to create naturally occurring plant and animal oils and exudate, such as vanilla. Synthetic and naturally derived flavour materials can then be blended into flavours by skilled flavourists for use in consumer products. Flavour materials can be obtained from specialist flavour suppliers, known as flavour houses, as individual chemicals, natural blends or as proprietary specialty blends where the full composition is not disclosed. The individual flavour materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994): “Perfume and Flavour Materials of Natural Origin”, S. Arctander, Ed., Elizabeth, N.J., 1960; and “Flavourings”, E. Ziegler and H. Ziegler (ed.), Wiley-VCH Weinheim, 1998. It will be understood that flavours can be volatile or have volatile components which are detected by the nose in the same way as fragrances. As such, flavour materials can also be classified according to their physical characteristics, such as volatility and hydrophobicity, using the methods described above for fragrance materials. Flavour materials can also be described according to their Hansen Solubility Parameters using the methods described above for fragrance materials.

Sources of flavour materials include essential oils, concretes, absolutes, resins, resinoids, balsams, and tinctures. Preferred flavour materials include anise oil, ethyl-2-methyl butyrate, vanillin, cis-3-heptenol, cis-3-hexenol, trans-2-heptenal, butyl valerate, 2,3-diethyl pyrazine, methylcyclo-pentenolone, benzaldehyde, valerian oil. 3,4-dimeth-axyphenol, amyl acetate, amyl cinnamate, y-butyryl lactone, trimethyl pyrazine, phenyl acetic acid, isovaleraldehyde, ethyl maltol, ethyl vanillin, ethyl valerate, ethyl butyrate, cocoa extract, coffee extract, peppermint oil, spearmint oil, clove oil, anethol, cardamom oil, wintergreen oil, cinnamic aldehyde, ethyl-2-methyl valerate, g-hexenyl lactone, 2,4-decadienal. 2,4-heptadienal, methyl thiazole alcohol (4-methyl-5-b-hydroxyethyl thiazole), 2-methyl butanethiol, 4-mercapto-2-butanone, 3-mercapto-2-pentanone, 1-mercapto-2-propane, benzaldehyde, furfural, furfuryl alcohol, 2-mercapto propionic acid, alkyl pyrazine, methyl pyrazine, 2-ethyl-3-methyl pyrazine, tetramethyl pyrazine, polysulfides, dipropyl disulphide, methyl benzyl disulphide, alkyl thiophene, 2,3-dimethyl thiophene, 5-methyl furfural, acetyl furan, 2,4-decadienal, guiacol, phenyl acetaldehyde, b-decalactone, d-limonene, acetoin, amyl acetate, maltol, ethyl butyrate, levulinic acid, piperonal, ethyl acetate, n-octanal, n-pentanal, n-hexanal, diacetyl, monosodium glutamate, monopotassium glutamate, sulfur-containing amino acids, e.g., cysteine, 2-methylfuran-3-thiol, 2-methyldihydrofuran-3-thiol, 2,5-dimethylfuran-3-thiol, tetramethyl pyrazine, propylpropenyl disulphide, propylpropenyl trisulfide, diallyl disulphide, diallyl trisulfide, dipropenyl disulphide, dipropenyl trisulfide. 4-methyl-2-[(methylthio)-ethyl]-1,3-dithiolane. 4,5-dimethyl-2-(methylthiomethyl)-1,3-dithiolane, and 4-methyl-2-(methylthiomethyl)-1,3-dithiolane, hop oils, and citrus oils such as lemon, orange, lime, and grapefruit.

As used herein, the term “butter” refers to a lipophilic fatty compound with a reversible solid/liquid state change and comprising a liquid fraction and a solid fraction at the temperature of 25° C., and at atmospheric pressure (760 mmHg). Preferred butters include lanolin and its derivatives such as lanolin alcohol, oxyethylenated lanolines, acetylated lanolin, lanolin esters such as isopropyl lanolate, oxypropylenated lanolines; polymeric or non-polymeric silicone compounds, such as polydimethylsiloxanes of high molecular weight, poly-dimethylsiloxanes with side chains of the alkyl or alkoxy type having from 8 to 24 carbon atoms, especially stearyl dimethicones; and vinyl polymers.

Preferably, the butter is of plant origin, such as those described in Ullmann's Encyclopaedia of Industrial Chemistry (“Fats and Fatty Oils”, A. Thomas, published on June 2000). Examples include triglycerides C10-C18 comprising a liquid fraction and a solid fraction at a temperature of 25° C., and at atmospheric pressure (760 mm Hg), shea butter, Nilotica Shea butter, Galam butter, Borneo butter or Tengkawang tallow, Shorea butter, Illipé butter, Madhuca butter or Bassia Madhuca longifolia, mowrah butter, Katiau butter, Phulwara butter, mango butter, Murumuru butter, Kokum butter, Ucuuba butter, Tucuma butter, Painya butter, Coffee butter, Apricot butter, Macadamia butter, grape butter, avocado butter, olive butter, sweet almond butter, cocoa butter, sunflower butter, Astrocaryum murumuru Seed Butter, Theobroma Grandiflorum Seed Butter, Irvingia Gabonensis Kernel Butter, jojoba esters (mixture of wax and oil hydrogenated jojoba) and ethyl esters of shea butter, and combinations thereof.

As used herein, the term “wax” refers to a lipophilic compound that is solid at 25° C., with a reversible solid/liquid state change, having a melting point greater than or equal to 30° C. up to 120° C.

Examples of waxes include hydrocarbon-based waxes such as beeswax, lanolin wax, and Chinese insect waxes, rice wax, Carnauba wax, Maydella wax, Ouricurry wax, Alfa wax, cork fiber wax, sugar maye wax, Japanese wax and sumac wax; montan wax, microcrystalline waxes, paraffins and ozokerite: polyethylene waxes, waxes obtained by Fisher-Tropsch synthesis and waxy copolymers and their esters, and mixtures thereof.

Further examples include waxes obtained by catalytic hydrogenation of animal or vegetable oils having linear or branched C8-C32 fatty chains. Among these are hydrogenated jojoba oil, hydrogenated sunflower oil, hydrogenated castor oil, hydrogenated coconut oil and hydrogenated lanolin oil, di-tetrastearate (trimethylol-1,1,1 propane), di-(1,1,1-trimethylolpropane) tetraprenate.

Further examples include the waxes obtained by transesterification and hydrogenation of vegetable oils, such as castor oil or olive oil.

Further examples include silicone waxes, e.g. polysiloxanes. Among the commercial silicone waxes of this type, mention may be made in particular of those sold under the names Abilwax 9800, 9801 or 9810 (GOLDSCHMIDT), KF910 and KF7002 (SHIN ETSU), or 176-1118-3 and 176-11481 (GENERAL ELECTRIC). The silicone waxes that may be used may also be alkyl or alkoxydimethicones such as the following commercial products: Abilwax 2428, 2434 and 2440 (GOLDSCHMIDT), or VP 1622 and VP 1621 (WACKER), as well as (C20-C60) alkyidimethicones, in particular especially the (C30-C45) alkyldimethicones such as the silicone wax sold under the name SF-1642 by GE-Bayer Silicones.

### DETAILED DESCRIPTION OF THE INVENTION

The inventors of the present invention have uncovered a method to prepare a plant-based protein colloidal dispersion into which a lipophilic phase, optionally containing an active ingredient(s), can be dispersed. The method allows for the formation of an oil-in-water emulsion that has excellent long-term physical stability, with the added benefit that it has a much lower potential to elicit an allergic response than conventional emulsifiers and so can be employed in a wide range of consumer product applications.

The emulsions of the present invention contain small, substantially insoluble plant protein particles that stabilise the lipophilic phase by a Pickering mechanism (i.e. the emulsions are Pickering emulsions) in the absence of any conventional amphiphilic emulsifiers.

The present invention is directed to a method for the preparation of an emulsion, the method comprising
(a) forming a solution comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s);
(b) inducing the protein in the solution to undergo a sol-gel transition to form a plant-based protein hydrogel;
(c) subjecting the plant-based protein hydrogel to a first shear step to form a first plant-based protein hydrogel slurry;
(d) subjecting the first plant-based protein hydrogel slurry to a solvent reduction step to form a washed plant-based protein hydrogel slurry;
(e) subjecting the washed plant-based protein hydrogel slurry to a second shear step to form a second plant-based protein hydrogel slurry; and
(f) dispersing a lipophilic phase in the second plant-based protein hydrogel slurry to form the emulsion,
wherein said emulsion has a protein solids content of less than 1 wt % based upon the total weight of the emulsion.

Any suitable plant-based proteins may be used in the present invention. In preferred methods of the present invention, the plant-based protein(s) is obtained from fava bean, mung bean, pea, rice, potato, rapeseed, lentil, chickpea, sunflower seed, pumpkin seed, flax, chia, canola, lupine, alfalfa, moringa, wheat, corn zein or sorghum; preferably the plant-based protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein. More preferably, the plant-based protein is pea protein and/or potato protein. Such proteins are considered to be low allergenicity proteins.

Suitable plant-based proteins further include:
Brassicas: including Brassica balearica: Mallorca cabbage, Brassica carinata; Abyssinian mustard or Abyssinian cabbage, Brassica elongata: elongated mustard, Brassica fruticulosa: Mediterranean cabbage, Brassica hilarionis: St Hilarion cabbage, Brassica juncea: Indian mustard, brown and leaf mustards, Sarepta mustard. Brassica napus: rapeseed, canola, rutabaga, Brassica narinosa: broadbeaked mustard, Brassica nigra: black mustard, Brassica oleracea: kale, cabbage, collard greens, broccoli, cauliflower, kai-lan. Brussels sprouts, kohlrabi, Brassica perviridis: tender green, mustard spinach, Brassica rapa (syn. B, campestris): Chinese cabbage, turnip, rapini, komatsuna, Brassica rupestris: brown mustard. Brassica tournefortii: Asian mustard
Solanaceae: including tomatoes, potatoes, eggplant, bell and chili peppers;
cereals: including maize, rice, wheat, barley, sorghum, millet, oats, rye, triticale, fonio
pseudocereals: including amaranth (love-lies-bleeding, red amaranth, prince-of-Wales-feather), breadnut, buckwheat, chia, cockscomb (also called quail grass or soko), pitseed Goosefoot, qañiwa, quinoa and, wattleseed (also called acacia seed);
Legume: including Acacia alata (Winged Wattle), Acacia decipiens. Acacia saligna (commonly known by various names including coojong, golden wreath wattle, orange wattle, blue-leafed wattle), Arachis hypogaea (peanut), Astragalus galegiformis, Cytisus laburnum (the common laburnum, golden chain or golden rain), Cytisus supinus, Dolichios lablab (common names include hyacinth bean, lablab-bean bonavist bean/pea, dolichos bean, seim bean, lablab bean, Egyptian kidney bean, Indian bean, bataw and Australian pea.), Ervum lens (Lentil), Genista tinctorial (common names include dyer's whin, waxen woad and waxen wood), Glycine max (Soybean). Lathyrus clymenum (peavines or vetchlings), Lathyrus odoratus (peavines or vetchlings), Lathyrus staivus (peavines or vetchlings), Lathyrus silvetris (peavines or vetchlings), Lotus tetragonolobus (asparagus-pea or winged pea), Lupinus albus (Lupin), Lupinus angustifolius (lupin), Lupinus luteus (Lupin), Lupinus polyphyllus (Lupin). Medicago sativa (Alfalfa), Phaseolus aureus (Mung bean), Phaseolus coccineus (Runner bean), Phaseolus nanus (Green bean/French bean), Phaseolus vulgaris (Green bean/French bean), Pisum sativum (pea). Trifolium hybridum (Clover). Trifolium pretense (Red clover). Vicia faba (Broad bean), Vicia sativa (Vetch). Vigna unguiculate (cowpea)
Non-Legumes including: Acanshosicyos horrida (Acanshosicyos horrida). Aesculus hyppocastanum (Conker tree/Horsechestnut), Anacardium occidentale (Cashew tree), Balanites aegyptica, Bertholletia excels (Brazil nut), Beta vulgaris (Sugar beet). Brassica napus (Rapeseed), Brassica juncea (Brown mustard), Brassica nigra (Black mustard), Brassica hirta (Eurasian mustard). Cannabis sativa (marijuana), Citrullus vulgaris (Sort of watermelon), Citrus aurantiaca (Citrus). Cucurbita maxima (squash), Fagopyrum esculentum (knotweed), Gossypium barbadense (Extra long staple cotton), Heianthus annuus (sunflower), Nicotiana sp. (Tobacco plant), Prunus avium (cherry), Prunus cerasus (Sour cherry), Prunus domestica (plum), Prunus amygdalus (almond), Rricinus communis (Caster bean/caster oil plant), Sasamum indicum (Sesame), Sinapis alba (White mustard), Terlfalrea pedata (Oyster nul).

For the avoidance of doubt, the plant-based emulsions of the present invention do not encompass plants in their natural state.

In step (a), the first co-solvent increases solubility of the plant-based protein(s). The first co-solvent may be considered a solubilising co-solvent. There may be one or more solubilising co-solvent(s) and the solubilising co-solvent(s) may fully or partially solubilise the plant-based protein(s).

Examples of solubilising co-solvents are organic acids. An organic acid is an organic compound with acidic properties. Preferably, the organic acids are sourced from natural plant-based or bio-based feed-stocks.

In preferred methods of the present invention, the first co-solvent is an organic acid. Preferably, the organic acid is acetic acid, lactic acid, formic acid, gluconic acid, propionic acid, an α-hydroxy acid and/or a β-hydroxy acid. Preferred α-hydroxy acids include glycolic acid, acetic acid, lactic acid, malic acid, citric acid and/or tartaric acid, preferably acetic acid and lactic acid. Preferred β-hydroxy acid may include β-hydroxypropionic acid, β-hydroxybutyric acid, β-hydroxy β-methylbutyric acid, 2-hydroxybenzoic acid and carnitine. In particularly preferred methods of the present invention, the organic acid is acetic acid or lactic acid.

Using an organic acid enables solubilisation of the plant protein and also allows for mild hydrolysis of the protein. For example, without wishing to be bound by theory, the solubility of plant-based proteins in organic acid is possible due to: i) the protonation of proteins and ii) the presence of an anion solvation layer which contributes to a reduction of hydrophobic interactions. Once initially dissolved in organic acid, the protonation of plant-based proteins can help to stabilise them in its non-solvent, for example water.

In step (a), the second co-solvent has decreased solubility of the plant based protein(s), as compared to the first co-solvent. The second co-solvent may be considered a de-solubilising co-solvent. There may be one or more de-solubilising co-solvent(s).

In preferred methods of the present invention, the second co-solvent is selected from water, ethanol, and/or ethyl acetate, more preferably water and/or ethanol, even more preferably water.

In preferred methods of the present invention, the solvent system has a co-solvent ratio of first co-solvent to second co-solvent of about 10-90% v/v, preferably 20-90% v/v, preferably about 20-80% v/v, preferably about 20-60% v/v, about 25-55% v/v, about 30-50% v/v, about 20%, about 30%, about 40%, about 50% or about 60% v/v, most preferably about 30-50% v/v.

In preferred methods of the present invention, the concentration of the plant-based protein(s) in the solvent system is 25-200 mg/ml, more preferably 50-150 mg/ml. The ratio of organic acid may vary depending on protein concentration, e.g. using a higher organic acid ratio with increasing protein concentration.

In preferred methods of the present invention, the degree of protein hydrolysis (i.e, the percentage of cleaved peptide bonds in a protein hydrolysate) is controlled to modify the properties of the resultant hydrogel. For example, increasing the acid concentration present during formation will increase the degree of protein hydrolysis. Higher degree of protein hydrolysis leads to the formation of less rigid hydrogels.

In preferred methods of the present invention, the degree of protein hydrolysis is 0.1 to 10%, preferably 0.1 to 5%, even more preferably 0.1 to 2.5%.

In order to form the solution comprising one or more plant-based protein(s), it may be necessary to apply physical stimulus to the protein/solvent system mixture to enable dissolution of the protein. Suitable physical stimulus includes heating, ultrasonication, agitation, high-shear mixing, high-shear homogenisation or other physical techniques. A preferred technique is heating, optionally with subsequent ultrasonication.

Preferably, the protein/solvent system mixture is subjected to a physical stimulus which is heating, wherein the solution is heated to about or above 70° C. More preferably, the protein/solvent system mixture is heated to about or above 75° C., about or above 80° C., about or above 85° C., or about 90° C. Even more preferably, the protein/solvent system mixture is heated to 85° C.

Preferably, the protein/solvent system mixture is subjected to a physical stimulus which is heating for a period of about 5 minutes, 10 minutes, 15 minutes, 20 minutes, 25 minutes, 30 minutes, or greater than 30 minutes. The heated protein/solvent system mixture is optionally subjected to subsequent ultrasonication.

In preferred methods of the present invention, in step (b) the protein solution is heated to a first temperature above the sol-gel transition temperature of the one or more plant-based protein(s) solution, then reduced to a second temperature below the sol-gel transition temperature of the one or more plant-based protein(s) solution to form a hydrogel.

The protein solution is heated such that the liquid solution is held above the sol-gel transition for the protein(s). By modifying the solvent system (for example through selection of the choice of organic acid, the ratio of organic acid to further solvent or through further means) it is possible to modify the sol-gel transition temperature for the protein(s). Through appropriate selection of conditions, it is possible to carefully control the sol-gel transition of the protein thereby controlling the formation of the hydrogel.

Preferably, the protein solution is heated to about or above 70° C. More preferably, the protein is heated to about or above 75° C., about or above 80° C., about or above 85° C., or about 90° C. Even more preferably, the protein is heated to 85° C.

The protein solution may be held at elevated temperature for a time period of about 5 minutes, 10 minutes, 15 minutes, 20 minutes, 25 minutes. 30 minutes, 45 minutes or 1 hour. A preferred time period is at least 30 minutes to enable the proteins to fully solubilise. It is possible to hold the protein solution at an elevated temperature for a longer period of time.

Having heated the protein solution to above the sol-gel transition temperature, the temperature of the protein solution can be reduced to a second temperature below the sol-gel transition temperature to facilitate formation of the hydrogel. The second temperature may be room temperature. The second temperature may be in the range 5 to 25° C., preferably 10 to 20° C. The protein solution may be held at the reduced temperature for long periods of time, e.g. days, weeks prior to performing the first shear step in step (c). The protein solution may be held at the reduced temperature for a time period of about 5 minutes, 10 minutes, 15 minutes, 20 minutes, 25 minutes or about 30 minutes. A particular reduced time period is about 5 minutes.

The particular temperatures will depend on the properties of the protein source, the solvent conditions used and therefore the sol-gel transition temperature. Alternatively, the elevated and reduced temperatures may be relatively fixed (for example about 85° C., then about room temperature) and the co-solvent mixture conditions are adjusted to ensure a suitable sol-gel transition temperature for the selected plant-based protein.

Without wishing to be bound by theory, it is believed that when the plant protein is added to the solvent system the plant protein forms a dispersion of insoluble colloidal protein aggregates. Aggregate size may be measured by Dynamic Light Scattering (DLS). Suitable apparatus to measure aggregate size is a Zetasizer Nano S (Malvern).

It is believed that upon heating the protein solution in the presence of a co-solvent system to above the sol-gel transition temperature, the plant proteins partially unfold, resulting in the exposure of hydrophobic amino acids initially buried within the protein native structure. Once partially unfolded, the co-solvents are able to interact with the unfolded protein molecules. For example, an organic acid has greater access to protonate amino acid residues, as well as enabling the formation of anion salt bridges that stabilise hydrophobic interactions. Also, upon heating at elevated temperatures, protein-protein non-covalent intermolecular contacts are disrupted.

Further, it is believed that the application of mechanical agitation, for example ultrasonication, disrupts large colloidal protein aggregates into smaller ones, as well as disrupting protein intermolecular interactions. Using this approach, the size of the protein aggregates can be significantly reduced, before gelation, to particle sizes below 100 nm. Preferably, the method described above comprises protein aggregates with an average size less than 200 nm, preferably less than 150 nm, less than 125 nm, less than 100 nm, less than 90 nm, less than 80 nm, less than 70 nm, less than 60 nm, less than 50 nm, less than 40 nm, or less than 30 nm. It is therefore thought that at this stage in the method the plant-based protein(s) have protein secondary structures with high levels of α-helix and random coil.

Further, it is believed that upon cooling the protein solution to below the sol-gel transition temperature, protein-protein non-covalent intermolecular contacts are enabled, thus promoting the self-assembly of plant protein molecules into a hydrogel of inter-connected protein aggregates.

After gelation, the aggregates may be fine stranded. The aggregates may have a median average length of between 50 to 500 nm. The aggregates may have a mean average length of between 50 to 500 nm. 80% of the aggregates may have an average length of between 50 to 500 nm. The aggregates may have a median height of between to 50 nm. The aggregates may have a mean average height of between 5 to 50 nm. 80% of the aggregates may have an average height of between 5 to 50 nm. In a preferred embodiment, the aggregates have a median average length of between 50 to 500 nm and/or a median average height of between 5 to 50 nm.

It is believed that the method of the present invention allows the plant proteins to aggregate into supramolecular structures held by intermolecular hydrogen bonding interactions, and in particular between the β-strands.

The methods of the present invention enable materials to be formed in which there are high levels of β-sheet intermolecular interactions. Thus, in methods of the present invention, the plant-based protein(s) have a protein secondary structure with at least 40% intermolecular β-sheet, at least 50% intermolecular β-sheet, at least 60% intermolecular β-sheet, at least 70% intermolecular β-sheet, at least 80% intermolecular β-sheet, or at least 90% intermolecular 3-sheet, wherein the % intermolecular β-sheet content is measured by FTIR (Fourier transform infrared spectroscopy).

In the method of the present invention, step (c) involves a first shear step. The first shear step may be a lower shear step.

In preferred methods of the present invention, said first shear step involves fragmenting the plant-based protein hydrogel into fragments. Preferably, at least 50 wt % of said fragments produced in said first shear step have a particle dimension in the range 1 mm to 100 mm, preferably 1 mm to 50 mm, preferably 5 mm to 30 mm, more preferably 10 mm to 30 mm. More preferably, at least 80 wt % of said fragments produced in said first shear step have a particle dimension in the range 1 mm to 100 mm, preferably 1 mm to 50 mm, preferably 5 mm to 30 mm, more preferably 10 mm to 30 mm. This can be measured by optical microscopy.

In preferred methods of the present invention, the first shear step is conducted at a temperature that is below the sol-gel transition temperature of the plant-based protein(s).

In preferred methods of the present invention, said first shear step involves mechanical cutting. By mechanical cutting, we mean cutting using a knife edge (e.g. a knife, an extruder blade etc.)

In alternative preferred methods of the present invention, said first shear step involves extrusion. For example, the plant-based protein solution formed in step (a) can be extruded into a non-solubilising solvent (e.g. water) to form the plant-based protein hydrogel in large discrete fragments, e.g. the large discrete fragments may take the form of extrudates having a thread or string form. In this way, the fragments can be directly subjected to a solvent reduction step, as described in more detail below. A first shear step of this nature is more amenable to large scale processing. In this case, the first shear step may reduce the at least one dimension of the large fragment to between 1 mm and 100 mm, for example a diameter of the extrudate. Preferably, at least 50 wt % of said fragments produced in said first shear step have at least one internal dimension in the range 1 mm to 100 mm, preferably 1 mm to 50 mm, preferably 5 mm to 30 mm, more preferably 10 mm to 30 mm. More preferably, at least 80 wt % of said fragments produced in said first shear step have at least one internal dimension in the range 1 mm to 100 mm, preferably 1 mm to 50 mm, preferably 5 mm to 30 mm, more preferably 10 mm to 30 mm. This can be measured by optical microscopy.

In the methods of the present invention, step (d) involves subjecting the first plant-based protein hydrogel slurry to a solvent reduction step, preferably a solubilising solvent reduction step.

By solubilising solvent, we mean a solvent or mixture of solvents in which the plant-based protein hydrogel dissolves. Examples include organic acids: such as acetic acid, lactic acid, formic acid, propionic acid, an α-hydroxy acid and/or a β-hydroxy acid. The α-hydroxy acid may preferably be selected from glycolic acid, acetic acid, lactic acid, malic acid, citric acid and/or tartaric acid. The β-hydroxy acid may preferably be selected from β-hydroxypropionic acid. β-hydroxybutyric acid, β-hydroxy β-methylbutyric acid, 2-hydroxybenzoic acid and carnitine.

In preferred methods of the present invention, said solvent reduction step comprises the steps of:
(i) contacting the first plant-based protein hydrogel slurry with a non-solubilising solvent;
(ii) separating the first plant-based hydrogel slurry from the non-solubilising solvent to give a washed plant-based protein hydrogel slurry; and
(iii) optionally repeating steps (i) and (ii).

Step (i) involves contacting the first plant-based protein hydrogel slurry with a non-solubilising solvent. By non-solubilising solvent, we mean a solvent or mixture of solvents in which the plant-based protein hydrogel does not dissolve. Examples include water or a mixture of water and ethanol.

In preferred methods of the present invention, step (ii) involves mesh filtration or centrifugation. More preferably, step (ii) involves mesh filtration using multiple meshes of decreasing size.

As would be understood by a skilled person, if the fragments produced in the first shear step are too small the solvent reduction step can prove difficult as the fragments can end up blocking the meshes or the collected yield is low. However, if the fragments produced in the first shear step are too large, the solvent reduction step can take excessive amounts of time due to the slow mass transport of solvent from the core of the fragments.

Without wishing to be bound by theory, it is thought that due to the porous nature of the hydrogel, the solvent reduction step can remove some or all of the solvent (e.g. organic acid) from the hydrogel via a solvent exchange.

The strength of a protein hydrogel can be altered by varying the concentration of protein and organic acid, amongst other variables.

In the method of the present invention, step (e) involves a second shear step to produce a second plant-based protein hydrogel slurry. The second shear step may be a higher shear step.

In preferred methods of the present invention, said second shear step involves further fragmenting the plant-based protein hydrogel.

In preferred methods of the present invention, said second plant-based protein hydrogel slurry has a de of less than 100 μm as measured by laser diffraction, preferably less than 50 μm, more preferably less than 30 μm, more preferably less than 10 μm, more preferably less than 5 μm, more preferably less than 1 μm.

In preferred methods of the present invention said second plant-based protein hydrogel slurry has a de of less than 250 μm as measured by laser diffraction, preferably less than 100 μm, more preferably less than 50 μm, more preferably less than 30 μm, more preferably less than 10 μm.

In preferred methods of the present invention, the particle size distribution of the hydrogel fragments in the second plant-based protein hydrogel slurry can be adjusted by varying the nature and the intensity of the second shear step. In another preferred method, the particle size distribution of the hydrogel fragments in the second plant-based protein hydrogel slurry can be adjusted by blending or combining two or more different hydrogel slurries that have been subjected to different second shear steps and having different particle size distributions.

In preferred methods of the present invention, said second shear step is conducted at a temperature that is below the sol-gel transition temperature of the plant-based protein(s).

In preferred methods of the present invention, said second shear step is conducted at a temperature that is below the protein denaturation temperature of the plant-based protein(s).

In preferred methods of the present invention, said second shear step is conducted for a duration of at least 5 minutes, more preferably at least 1 minute.

In preferred methods of the present invention, said second shear step involves ultrasonication (e.g. using equipment such as a Bandelin HD4200 or a Hielscher UIP1000hdT), high-shear mechanical stirring (e.g. using equipment such as a Silverson rotor-stator high-shear mixer), high pressure homogenisation, or cavitation, preferably ultrasonication.

In preferred methods of the present invention, said second shear step involves one or more steps, preferably two steps.

In preferred methods of the present invention, the protein solids of the second plant-based protein hydrogel slurry has a biodegradation percentage based upon Oz consumption as measured according to ISO-14851 version 2019 after 28 days of 60 to 100% based upon the ratio of the Biological Oxygen Demand (BOD) to the Theoretical Oxygen Demand, more preferably 65 to 100%, even more preferably 70 to 100%, even more preferably 75 to 100%, even more preferably 80 to 100%, even more preferably 85 to 100%, most preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

In preferred methods of the present invention, said second plant-based protein hydrogel slurry is subjected to a pH adjustment step between steps (e) and (f).

In preferred methods of the present invention, said pH adjustment step involves adding a pH-modification material to the second plant-based protein hydrogel slurry. Preferably, said pH-modification material is a solution comprising monovalent metal ions, divalent metal ions or ammonium ions, preferably an aqueous alkaline solution comprising monovalent metal ions, divalent metal ions or ammonium ions. More preferably, said pH-modification material is an aqueous hydroxide solution, preferably sodium hydroxide, potassium hydroxide, or ammonium hydroxide.

In preferred methods of the present invention, said lipophilic phase comprises a solvent, a butter or a wax.

In preferred methods of the present invention, the lipophilic phase comprises a solvent. Preferably, the solvent is a solvent with low volatility (e.g. having a vapour pressure of less than 0.1 Torr at 25° C., preferably less than 0.01 Torr at 25° C., preferably less than 0.001 Torr at 25° C.).

Preferably, the solvent has low or no odour.

Preferably, the solvent has at least two Hansen solubility parameters selected from: an atomic dispersion force (OD) of less than 20, a dipole moment (δP) of less than 8, and a hydrogen bonding (8H) of less than 11 More preferably, the solvent has at least two Hansen solubility parameters selected from, an atomic dispersion force (δD) of less than 20, a dipole moment (δP) of less than 4, and a hydrogen bonding (δH) of less than 5.

Preferably, the solvent contains only low levels of materials with an alcohol functionality (e.g. a primary alcohol functionality). In preferred methods of the present invention, the solvent comprises less than 40% wt alcohol-containing material based upon the total weight of the solvent, more preferably less than 20% wt. In particularly preferred methods of the present invention, the solvent does not comprise an alcohol-containing material. Without wishing to be bound by theory, it is thought that alcohols, and particularly primary alcohols having a straight chain alkyl group, can easily degrade an emulsion.

In preferred methods of the present invention, said lipophilic phase comprises a solvent selected from a fatty acid ester, a fatty acid, a linear or branched hydrocarbon of natural mineral or synthetic origin, a fatty alcohol or ether thereof, a vegetable oil, a silicone oil, a phthalate ester, a rosin resin, a diol, a triol, benzyl benzoate, triethyl citrate and triacetin, or combinations thereof.

Preferably, the solvent is selected from Miglyol® 840, Miglyol® 812 N, Miglyol® 829. Miglyol® 829 ECO, Miglyol® Coco 810, Miglyol®) 810 N. Miglyoi® 128, Miglyol® 808, Miglyok® T-C7, Miglyol® 8810, Miglyol®) PPG 810, Miglyol® OE, Miglyo® DO, and Miglyok? 818. Abalyn®, limonene, benzyl benzoate, diethyl phthalate, isopropyl myristate, triethyl citrate, dipropylene glycol, and propylene glycol, triacetin, glycerin. 1,3 propanediol or combinations thereof, preferably Miglyol® 812 N.

Preferably, the solvent is a vegetable oil selected from coconut oil, corn oil, canola oil, cottonseed oil, olive oil, palm oil, peanut oil, rapeseed oil, safflower oil, sesame oil, soybean oil, and sunflower oil, squash oil, grape seed oil, hazelnut oil, apricot oil, macadamia oil, avocado oil, meadowfoam seed oil Limnanthes Alba, Macadamia nut oil, Rosehip rose oil, apricot kernel oil, rice bran oil, argan oil, evening primrose oil, palm oil, rice germ oil, sweet almond oil, peanut seed oil, oil of Mortierella isabelline, safflower seed oil, Queensland nut oil, Macadamia Integrifolia Seed Oil, wheat germ oil, borage seed oil, shea oil, hazelnut oil), oil of mango seed, pomegranate seed oil, Chinese cabbage seed oil, passion fruit seed oil, camellia seed oil of Japan, green tea seed oil, corn germ oil, oil of hoplostete. Brazil nut oil, musk rose seed oil, Inca seed Inchi oil, Babassu seed oil, sea buckthorn oil, Marula seed oil, baobab seed oil, baobab oil, Moringa seed oil, castor seed oil, black currant seed oil, tea seed oil, raspberry seed oil, Abyssinian crambe seed oil, rosehip seed oil, tomato seed oil, bitter almond oil, yuzu seed oil, pumpkin seed oil, desert date seed oil, Japanese white pine seed oil, watermelon seed oil, walnut seed oil, nigella oil, carrot seed oil, cranberry seed oil, vanilla oil, cranberry seed oil, Acai oil, peach kernel-core oil, glycerides from mayola oil and phytosterols, karanja seed oil and roucou oil, rosehip oil, coriander oil, linseed oil, chia oil. Fenugreek oil, and hemp oil, or combinations thereof.

Preferably, the solvent is a hydrogenated vegetable oil selected from hydrogenated palm oil, hydrogenated coconut oil, hydrogenated rapeseed oil, hydrogenated castor oil, hydrogenated palm kernel oil, triester of hydrogenated castor oil and isostearic acice, hydrogenated cottonseed oil, hydrogenated olive oil, hydrogenated peanut oil, and hydrogenated soybean oil, or combinations thereof.

A vegetable oil is an oil that comes from plant sources. Alternatively, the solvent is derived from a vegetable oil. As will be understood by a skilled person, vegetable oil solvent or vegetable oil-derived solvent may provide additional benefit beyond its solvation properties, e.g. as a moisturiser in a cosmetic application.

Preferred fatty acid esters include the oils of formulas R1COOR2 and R1OR2 in which R1 represents the residue of a C8 to C29 fatty acid, while R2 represents a hydrocarbon chain, branched or unbranched, C3 to C30, such as, for example, purcellin oil, isononyl isononanoate, isodecyl neopentanoate, isopropyl myristate, 2-ethylhexyl palmitate, octyl-2 stearate dodecyl, octyl-2-dodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates, demayoates of fatty alcohols; polyol esters, such as propylene glycol dioctanoate, neopentyl glycol diheptanoate and diethylene glycol diisononanoate; and pentaerythritol esters such as pentaerythrityl tetrahehenate (DUB PTB) or pentaerythrityl tetraisostearate (Prisorine 3631), triglycerides such as triglycerides of caprylic, capric, myristic and stearic acids, triethylhexanoine, tribehenine, triisostearin, tricaprylin (or triacylglycerol), trihydroxymethoxystearin, and triheptanoine.

Preferred fatty acids include stearic acid, palmitic acid, myristic acid, lauric acid, capric acid, and caprylic acid.

Preferred linear or branched hydrocarbon of natural mineral or synthetic origin include paraffin oils, volatile or not, and their derivatives, petroleum jelly, polydecenes, hydrogenated polyisobutene such as Parleam oil.

Preferred silicone oils include volatile or non-volatile polymethylsiloxanes (PDMS) with a linear or cyclic silicone chain, which are liquid or pasty at room temperature, in particular cyclopolydimethylsiloxanes (cyclomethicones) such as cyclohexasiloxane and cyclopentasiloxane: polydimethylsiloxanes (or dimethicones) comprising alkyl, alkoxy or phenyl groups, during or at the end of the silicone chain, groups having from 2 to 24 carbon atoms: phenyl silicones such as phenyltrimethicones, phenyldimethicones, phenyltrimethylsiloxydiphenylsiloxanes, diphenyldimethi-cones, diphenylmethyldiphenyltrisiloxanes. 2-phenylethyltri-methylsiloxysilicates, and polymethylphenyl-siloxanes.

Preferred fatty alcohols include those having from 8 to 26 carbon atoms, such as cetyl alcohol, stearyl alcohol and their mixture (cetylstearyl alcohol), or octyldodemayol.

In preferred methods of the present invention, said lipophilic phase comprises a wax which is selected from Softisan® 100. Softisan® 142, and Softisan® 154, or combinations thereof.

In preferred methods of the present invention, said lipophilic phase comprises an active ingredient(s). Preferably, said active ingredient(s) is selected from a vitamin, a mineral, a flavour material, a fragrance material, a pro-flavour, a pro-fragrance, a flavour enhancer, a malodour counteractant, a nutraceutical, a probiotic, a pharmaceutical, an anti-microbial agent, an anti-viral agent, an anti-inflammatory agent, an antioxidant, a pesticide, a herbicide, a fertiliser, a fungicide, an insecticide, an animal repellent, an antiacne agent, an anti-ageing agent, a skin lightening agent, an emollient, a humectant (e.g. α-hydroxyacids or hyaluronic acid), an occlusive agent, a skin moisturizing agent, an antiperspirant or deodorant agent, a wrinkle control agent, a fabric softener active, a surface cleaning active, a skin conditioning agent, a hair conditioning agent, a sunscreen, a dye, a pigment, and an adhesive, or combinations thereof.

Preferably, the agrochemicals, whether pesticides, herbicide, fertilisers, fungicides, insecticides or animal repellents, are a natural alternative to synthetic materials and are based on plant extracts and/or plant essential oils (EOs) or components of essential oils, such as thymol. Preferably, the agrochemical is a bio-pesticide. Preferably the agrochemicals are suitable for use in formulations for plant care and production that can be certified as organic by organisations, such as the USDA (US Department of Agriculture) or Ecocert in Europe.

In particularly preferred methods of the present invention said active ingredient(s) is at least one fragrance material or flavour material. Preferably, the at least one fragrance material or flavour material is selected from an alcohol, an aldehyde a ketone, an ester, an ether, an acetate, an alkene, a nitrile, a nitrogenous heterocyclic compound, a sulfurous heterocyclic compound, and a Schiff base.

The fragrance materials and flavour materials employed in the present invention may be of natural origin (i.e. they are extracted from a natural source and are not synthetically modified in any way). Preferred fragrance materials or flavour materials of natural origin include nutmeg extract, cardamon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, jasmine extract, olibanum extract, rose extract, vanillin, coffee extract, hop oil or combinations thereof. Preferably, the fragrance materials or flavour materials of natural origin are plant-derived. The fragrance materials or flavour materials of natural origin may be used alone, or in combination, or in combination with synthetic fragrance materials.

In preferred methods of the present invention, the at least one fragrance material or flavour material has a vapour pressure greater than or equal to 0.0001 Torr at 25° C.

In preferred methods of the present invention, the at least one fragrance material or flavour material has a log P greater than or equal to 3.0, preferably greater than or equal to 3.5, more preferably greater than or equal to 4.0.

In preferred methods of the present invention, the at least one fragrance material or flavour material has at least two Hansen solubility parameters selected from, an atomic dispersion force (δD) from 14 to 20, a dipole moment (δP) of less than 8, and a hydrogen bonding (δH) from 2.5 to 11.

In preferred methods of the present invention, the at least one fragrance material or flavour material is part of a fragrance or flavour.

Preferably, the fragrance or flavour contains at least 20 wt % of fragrance material(s) or flavour material(s) with a log P greater than or equal to 3.0 based upon the total weight of the fragrance or flavour, more preferably greater than or equal to 3.5, more preferably greater than or equal to 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 30 wt %, more preferably at least 50 wt %, more preferably at least 70 wt %.

Preferably, the fragrance or flavour contains at least 10 wt % of fragrance material(s) or flavour material(s) or essential oils which have a biodegradation percentage based upon O₂ consumption as measured according to ISO-14861 version 2019 after 28 days of 60 to 100% based upon the ratio of the Biological Oxygen Demand (BOD) to the Theoretical Oxygen Demand, more preferably 65%, even more preferably 70 to 100%, even more preferably 75 to 100%, even more preferably 80 to 100%, even more preferably 85 to 100%, most preferably 90 to 100%. Due to their low aqueous solubility, fragrance or flavours can be added to the biodegradation test on an inert support according to the method in ISO 10634 version 2018: Water quality—Preparation and treatment of poorly water-soluble organic compounds for the subsequent evaluation of their biodegradability in an aqueous medium, in addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (D) from 14 to 20, a dipole moment (δP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality) In preferred methods of the present invention, the fragrance or flavour comprises less than 40% wt alcohol-containing material based upon the total weight of the fragrance or flavour, more preferably less than 20% wt. In particularly preferred methods of the present invention, the fragrance or flavour does not comprise an alcohol-containing material.

Preferably, the fragrance or flavour material is of high odour impact. This is advantageous as it ensures that even low levels of fragrance are perceived when released from the emulsions.

In alternative particularly preferred methods of the present invention, the active ingredient(s) is a vitamin or a mineral. Preferably, said active ingredient(s) is a vitamin or a mineral selected from Vitamin A, Vitamin B1, Vitamin B2, Vitamin B3, Vitamin B5, Vitamin B6, Vitamin B7. Vitamin B9. Vitamin B12, Vitamin C. Vitamin D, Vitamin E, Vitamin K, magnesium, sodium, potassium, zinc, iron, calcium, iodine, omega 3, folic acid, thiamin, riboflavin, niacin and phosphorous, or mixtures thereof. More preferably, said active ingredient is Vitamin D.

In preferred methods of the present invention, step (f) involves membrane emulsification, high-shear mechanical stirring, ultrasonication, high-shear mechanical stirring, and/or cavitation.

In preferred methods of the present invention, said second plant-based protein hydrogel slurry is diluted with water prior to step (f), preferably with deionised water.

In preferred methods of the present invention, the emulsion has a protein solids content of less than 0.9 wt % based upon the total weight of the emulsion, preferably less than 0.8 wt %, preferably less than 0.7 wt %, preferably less than 0.6 wt %, preferably less than 0.5 wt %, preferably less than 0.4 wt %, preferably less than 0.3 wt %, preferably less than 0.2 wt %, preferably less than 0.1 wt %.

In preferred methods of the present invention, the emulsion is substantially free of soluble protein(s).

In preferred methods of the present invention, the emulsion comprises at least 0.1 wt % lipophilic phase based upon the total weight of the emulsion, preferably at least 0.5 wt %.

Preferred methods of the present invention further comprise a step of altering the pH of the emulsion such that it is different to the isoelectric point of the plant-based protein by more than 1 pH unit, preferably by more than 1.5 pH units.

In preferred methods of the present invention, said step of altering the pH of the emulsion involves adding a pH-modification material to the emulsion. Preferred pH modification materials are described above.

In preferred methods of the present invention, the pH of the emulsion after said step of altering the pH of the emulsion is below the isoelectric point of the plant-based protein by at least 1 pH unit, preferably by at least 1.5 pH units.

In alternative preferred methods of the present invention, said the pH of the emulsion after said step of altering the pH of the emulsion is above the isoelectric point of the plant-based protein by at least 1 pH unit, preferably by at least 1.5 pH units.

In preferred methods of the present invention, the step of altering the pH of the emulsion involves causing the plant-based protein to move through the isoelectric point. The isoelectric point of a particular plant-based protein can be determined using the method described in Helmick et al., Food Biophysics (2021) 16:474-483.

In preferred methods of the present invention, the step of altering the pH of the emulsion involves increasing the pH.

In preferred methods of the present invention, said the pH of the emulsion after said step of altering the pH of the emulsion is in the range 5.5 to 7.5, preferably 6.0 to 7.0. Such a pH range is particularly useful in cosmetic formulations, which will come into contact with the skin during use.

Without wishing to be bound by theory, it is thought that the protein hydrogel slurries of the present invention have an elongated fine-stranded morphology, meaning that they are able to easily assemble around the droplets of the lipophilic phase and form a stable emulsion. If the pH of the emulsion is altered as described above, it is thought that as the isoelectric point of the plant-based protein is approached, these fine-stranded particles become entangled with a high packing density to help form an even more stable emulsion. Furthermore, if the pH of the emulsion passes through the isoelectric point of the plant-based protein at a slow enough rate, increased aggregation is able occur, which also contributes to an increased emulsion stability.

The present invention also provides an emulsion obtained by or obtainable by the method hereinbefore described.

The present invention also provides an emulsion comprising a lipophilic phase dispersed in a plant-based protein hydrogel slurry comprising a plant-based protein(s), wherein said emulsion has a protein solids content of less than 1 wt % based upon the total weight of the emulsion.

The emulsions of the present invention are Pickering emulsions. However contrary to conventional Pickering emulsions, the Pickering emulsions of the present invention are not composed of nanoparticles (average diameter below 100 nm) which due to their very small size can pose human health safety concerns. In addition, most conventional Pickering emulsions are not plant-derived, many using inorganic particles such as fumed silica, which means they cannot be used in “clean-label” and organic products.

Preferred emulsions of the present invention have a protein solids content of less than 0.9 wt % based upon the total weight of the emulsion, preferably less than 0.8 wt %, preferably less than 0.7 wt %, preferably less than 0.6 wt %, preferably less than 0.5 wt %, preferably less than 0.4 wt %, preferably less than 0.3 wt %, preferably less than 0.2 wt %, preferably less than 0.1 wt %.

In preferred emulsions of the present invention, the plant-based protein(s) is obtained from fava bean, mung bean, pea, rice, potato, rapeseed, lentil, chickpea, sunflower seed, pumpkin seed, flax, chia, canola, lupine, alfalfa, moringa, wheat, corn zein or sorghum; preferably the plant-based protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein. More preferably, the plant-based protein is pea protein and/or potato protein. Such proteins are considered to be low allergenicity proteins.

In preferred emulsions of the present invention, the plant-based protein(s) has been pre-treated with an organic acid. Preferably, the organic acid is acetic acid, lactic acid, formic acid, gluconic acid, propionic acid, an α-hydroxy acid, and/or a β-hydroxy acid, preferably acetic acid or lactic acid. Preferred α-hydroxy acids include glycolic acid, acetic acid, lactic acid, malic acid, citric acid and/or tartaric acid, preferably acetic acid and lactic acid. Preferred β-hydroxy acid may include β-hydroxypropionic acid, β-hydroxybutyric acid. β-hydroxy β-methylbutyric acid, 2-hydroxybenzoic acid and carnitine. In particularly preferred emulsions of the present invention, the organic acid is acetic acid or lactic acid.

In preferred emulsions of the present invention, the plant-based protein hydrogel slurry has a de of less than 100 μm as measured by laser diffraction, preferably less than 50 μm, more preferably less than 30 μm, more preferably less than 10 μm, more preferably less than 5 μm, more preferably less than 1 μm.

In preferred emulsions of the present invention, the plant-based protein hydrogel slurry has a d₅₀ of less than 250 μm as measured by laser diffraction, preferably less than 100 μm, more preferably less than 50 μm, more preferably less than 30 μm, more preferably less than 10 μm.

In preferred emulsions of the present invention, the solid material of the plant-based protein hydrogel slurry has a biodegradation percentage based upon O₂ consumption as measured according to ISO-14851 version 2019 after 28 days of 60 to 100% based upon the ratio of the Biological Oxygen Demand (BOD) to the Theoretical Oxygen Demand, preferably 65 to 100%, more preferably 70 to 100%, more preferably 75 to 100%, more preferably 80 to 100%, more preferably 85 to 100%, even more preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

In preferred emulsions of the present invention, the lipophilic phase comprises a solvent, a butter or a wax.

In preferred emulsions of the present invention, the lipophilic phase comprises a solvent. Preferably, the solvent is a solvent with low volatility (e.g. having a vapour pressure of less than 0.1 Torr at 25° C., preferably less than 0.01 Torr at 25° C., preferably less than 0.001 Torr at 25° C.).

Preferably, the solvent has low or no odour.

Preferably, the solvent has at least two Hansen solubility parameters selected from: an atomic dispersion force (δD) of less than 20, a dipole moment (δP) of less than 8, and a hydrogen bonding (δH) of less than 11. More preferably, the solvent has at least two Hansen solubility parameters selected from: an atomic dispersion force (OD) of less than 20, a dipole moment (δP) of less than 4, and a hydrogen bonding (δH) of less than 5.

Preferably, the solvent contains only low levels of materials with an alcohol functionality (e.g. a primary alcohol functionality). In preferred emulsions of the present invention, the solvent comprises less than 40% wt alcohol-containing material based upon the total weight of the solvent, more preferably less than 20% wt. In particularly preferred emulsions of the present invention, the solvent does not comprise an alcohol-containing material.

In preferred emulsions of the present invention, the lipophilic phase comprises a solvent selected from a fatty acid ester, a fatty acid, a linear or branched hydrocarbon of natural mineral or synthetic origin, a fatty alcohol or ether thereof, a vegetable oil, a silicone oil, a phthalate ester, a rosin resin, a diol, a triol, benzyl benzoate, triethyl citrate and triacetin, or combinations thereof.

Preferred fatty acid esters, fatty acids, linear or branched hydrocarbon of natural mineral or synthetic origin, silicone oils, vegetable oils and fatty alcohols are described above.

Preferably, said solvent is selected from Miglyok® 840, Miglyol® 812 N, Miglyol® 829. Miglyol® 829 ECO, Miglyol® Coco 810, Miglyo® 810 N. Miglyol® 128, Miglyo® 808. Miglyol® T-C7. Miglyol® 8810, Miglyol® PPG 810, Miglyol® OE, Miglyol® DO, and Miglyol® 818. Abalyn®, limonene, benzyl benzoate, diethyl phthalate, isopropyl myristate, triethyl citrate, dipropylene glycol, and propylene glycol, triacetin, glycerin, 1.3 propanediol or combinations thereof, preferably Miglyol® 812 N.

In preferred emulsions of the present invention, the lipophilic phase comprises a wax which is selected from Softisan® 100. Softisan® 142, and Softisan® 154, or combinations thereof.

In preferred emulsions of the present invention, the lipophilic phase comprises an active ingredient(s). Preferably, the active ingredient(s) is selected from a vitamin, a mineral, a flavour material, a fragrance material, a pro-flavour, a pro-fragrance, a flavour enhancer, a malodour counteractant, a nutraceutical, a probiotic, a pharmaceutical, an anti-microbial agent, an anti-viral agent, an anti-inflammatory agent, an antioxidant, a pesticide, a herbicide, a fertiliser, a fungicide, an insecticide, an animal repellent, an antiacne agent, an anti-ageing agent, a skin lightening agent, an emollient, a humectant (e.g. α-hydroxyacids or hyaluronic acid), an occlusive agent, a skin moisturizing agent, an antiperspirant or deodorant agent, a wrinkle control agent, a fabric softener active, a surface cleaning active, a skin conditioning agent, a hair conditioning agent, a sunscreen, a dye, a pigment, and an adhesive, or combinations thereof.

Preferably, the agrochemicals, whether pesticides, herbicide, fertilisers, fungicides, insecticides or animal repellents, are a natural alternative to synthetic materials and are based on plant extracts and/or plant essential oils (EOs) or components of essential oils, such as thymol. Preferably, the agrochemical is a bio-pesticide. Preferably, the agrochemicals are suitable for use in formulations for plant care and production that can be certified as organic by organisations such as the USDA (US Department of Agriculture) or Ecocert in Europe.

In particularly preferred emulsions of the present invention said active ingredient(s) is at least one fragrance material or flavour material. Preferably, the at least one fragrance material or flavour material is selected from an alcohol, an aldehyde, a ketone, an ester, an ether, an acetate, an alkene, a nitrile, a nitrogenous heterocyclic compound, a sulfurous heterocyclic compound, and a Schiff base.

The fragrance materials and flavour materials employed in the present invention may be of natural origin (i.e they are extracted from a natural source and are not synthetically modified in any way) Preferred fragrance materials or flavour materials of natural origin include nutmeg extract, cardamon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, jasmine extract, olibanum extract, rose extract, vanillin, coffee extract, hop oil or combinations thereof. Preferably, the fragrance materials or flavour materials of natural origin are plant-derived. The fragrance materials or favour materials of natural origin may be used alone, or in combination, or in combination with synthetic fragrance materials.

In preferred emulsions of the present invention, the at least one fragrance material or flavour material has a vapour pressure greater than or equal to 0.0001 Torr at 25° C.

In preferred emulsions of the present invention, the at least one fragrance material or flavour material has a log P greater than or equal to 3.0, preferably greater than or equal to 3.5, more preferably greater than or equal to 4.0.

In preferred emulsions of the present invention, the at least one fragrance material or flavour material has at least two Hansen solubility parameters selected from: an atomic dispersion force (δD) from 14 to 20, a dipole moment (δP) of less than 8, and a hydrogen bonding (δH) from 2.5 to 11.

In preferred emulsions of the present invention, the at least one fragrance material or flavour material is part of a fragrance or flavour.

Preferably, the fragrance or flavour contains at least 20 wt % of fragrance material(s) or flavour material(s) with a log P greater than or equal to 3.0 based upon the total weight of the fragrance or flavour, more preferably greater than or equal to 3.5, more preferably greater than or equal to 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 30 wt %, more preferably at least 50 wt %, more preferably at least 70 wt %.

Preferably, the fragrance or flavour contains at least 10 wt % of fragrance material(s) or flavour material(s) or essential oils which have a biodegradation percentage based upon Oz consumption as measured according to ISO-14861 version 2019 after 28 days of 60 to 100% based upon the ratio of the Biological Oxygen Demand (BOD) to the Theoretical Oxygen Demand, preferably 65 to 100%, more preferably 70 to 100%, more preferably 75 to 100%, more preferably 80 to 100%, more preferably 85 to 100%, even more preferably 90 to 100%. Due to their low aqueous solubility, fragrance or flavours can be added to the biodegradation test on an inert support according to the method in ISO 10634 version 2018: Water quality-Preparation and treatment of poorly water-soluble organic compounds for the subsequent evaluation of their biodegradability in an aqueous medium. In addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (δD) from 14 to 20, a dipole moment (OP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality). In preferred methods of the present invention, the fragrance or flavour comprises less than 40% wt alcohol-containing material based upon the total weight of the fragrance or flavour, more preferably less than 20% wt. In particularly preferred emulsions of the present invention, the fragrance or flavour does not comprise an alcohol-containing material.

Preferably, the fragrance or flavour material is of high odour impact. This is advantageous as it ensures that even low levels of fragrance are perceived when released from the emulsions.

In alternative particularly preferred emulsions of the present invention, the active ingredient(s) is a vitamin or a mineral. Preferably, said active ingredient(s) is a vitamin or a mineral selected from Vitamin A, Vitamin B1, Vitamin B2. Vitamin B3, Vitamin B5. Vitamin B6, Vitamin B7. Vitamin B9. Vitamin B12, Vitamin C. Vitamin D, Vitamin E, Vitamin K, magnesium, sodium, potassium, zinc, iron, calcium, iodine, omega 3, folic acid, thiamin, riboflavin, niacin and phosphorous, or mixtures thereof. More preferably, said active ingredient is Vitamin D.

In preferred emulsions of the present invention, the plant-based protein hydrogel slurry comprises pea protein and the active ingredient is Vitamin D.

Preferred emulsions of the present invention comprise at least 0.1 wt % lipophilic phase based upon the total weight of the emulsion, preferably at least 0.5 wt %.

In preferred emulsions of the present invention, the pH of the emulsion is such that it is different to the isoelectric point of the plant-based protein by more than 1 pH unit, preferably by more than 1.5 pH units.

Thus, in preferred emulsions of the present invention, the pH of the emulsion is below the isoelectric point of the plant-based protein by at least 1 pH unit, preferably by at least 1.5 pH units.

In alternative preferred emulsions of the present invention, the pH of the emulsion is above the isoelectric point of the plant-based protein by at least 1 pH unit, preferably by at least 1.5 pH units.

In preferred emulsions of the present invention, the pH of the emulsion is in the range 5.5 to 7.5, preferably 6.0 to 7.0. Such a pH range is particularly useful in cosmetic formulations, which will come into contact with the skin during use.

In preferred emulsions of the present invention, the plant-based protein(s) have a protein secondary structure with at least 40% intermolecular β-sheet, at least 50% intermolecular 8-sheet, at least 60% intermolecular β-sheet, at least 70% intermolecular β-sheet, at least 80% intermolecular β-sheet, or at least 90% intermolecular β-sheet, wherein the % intermolecular β-sheet content is measured by FTIR.

The present invention is also directed to a composition comprising an emulsion prepared according to the method as hereinbefore described. Preferably, the composition is a cosmetic composition, a fragrance composition (e.g. a fine fragrance or a laundry scent booster), a beverage composition, an oral care composition, a pharmaceutical composition, or an agricultural composition.

The present invention is also directed to the use of a plant-based protein hydrogel slurry as an emulsifier in a composition. Preferably, the composition is a cosmetic composition, a fragrance composition (e.g. a fine fragrance or a laundry scent booster), a beverage composition, an oral care composition, a pharmaceutical composition, or an agricultural composition.

### BRIEF DESCRIPTION OF THE FIGURES

FIG. 1 shows the particle size distribution of the pea protein hydrogel slurries of Examples 1A, 1B and 1C and the pea protein mixture of Example 2A.

FIG. 2 shows the emulsion size distribution of the emulsions of Examples 3A, 3B, 3C and 3D.

FIG. 3 shows the emulsion of Example 3A (in triplicate) after having been subjected to stressed conditions.

FIG. 4 shows the emulsion of Example 3B (in triplicate) after having been subjected to stressed conditions.

FIG. 5 shows the emulsion of Example 3C (in triplicate) after having been subjected to stressed conditions.

FIG. 6 shows the emulsion of Example 3D (in triplicate) after having been subjected to stressed conditions.

FIG. 7 shows the emulsion size distribution of the emulsions of Examples 3E, 3F, 3G and 3H.

FIG. 8 shows the emulsion of Example 3E (in triplicate) after having been subjected to stressed conditions.

FIG. 9 shows the emulsion of Example 3F (in triplicate) after having been subjected to stressed conditions.

FIG. 10 shows the emulsion of Example 3G (in triplicate) after having been subjected to stressed conditions.

FIG. 11 shows the emulsion of Example 3H (in triplicate) after having been subjected to stressed conditions.

FIG. 12 shows the particle size distribution of the pea protein hydrogel slurries of Example 4.

FIG. 13 shows the emulsion size distribution of the emulsions of Examples 5A and 5B.

FIG. 14 shows the emulsion of Example 5A (in triplicate) after having been subjected to stressed conditions,

FIG. 15 shows the emulsion of Example 5B (in triplicate) after having been subjected to stressed conditions.

### EXAMPLES

### Materials

Pea Protein Isolate (PPI) (80% protein) was purchased from Cambridge Commodities Ltd.
Lactic acid (food-grade, 280%) was purchased from Cambridge Commodities Ltd.
Acetic acid (glacial 99%) was purchased from Fisher Scientific.
Sodium benzoate was purchased from Fisher Scientific.
Potassium hydroxide (>85%) was purchased from Sigma Aldrich.
Miglyok® 812N was purchased from IOI Oleochemical.
Hydrochloric acid was purchased from Sigma Aldrich.
Geraniol was purchased from Carvansons Ltd
Undecavertol was purchased from Carvansons Ltd.
Delta damascone was purchased from Carvansons Ltd.
Dodecane nitrile was purchased from was purchased from Carvansons Ltd.
Thymol was purchased from Fisher Scientific.

### Measurement Methods

### Viscosity

Viscosity measurements were made using an Anton Paar MCR 92 Rheometer using a plate and cone measurement geometry with a 50 mm plate and 1 degree angle and a constant shear of 64 s⁻¹ at 20° C. Measurement was taken within 1 hour of making the sample.

pH

pH measurements were made using a Mettler Toledo FiveEasy pH meter

### Particle Size

Particle size measurements were carried out using laser diffraction with an Anton Paar PSA 1190.

The measurements were carried out by diluting the plant-based protein hydrogel slurry in an aqueous solution with acetic acid or lactic acid adjusted to the same pH. The slurry was diluted to the required concentration in order to have the desired optical density (normally 5-15% obscuration) for the measurement. The d₅₀ quoted is for the volume distribution, de values for the volume distribution can also be obtained in this way using laser diffraction.

### Emulsion Size

Particle size measurements were carried out using laser diffraction with an Anton Paar PSA 1190.

The measurements were carried out by diluting the emulsion in water adjusted to the same pH to the required concentration in order to have the desired optical density (normally 5-15% obscuration) for the measurement. The de quoted is for the volume distribution, d₅₀ values for the volume distribution can also be obtained in this way using laser diffraction.

### Protein Solids Content

Protein solids content was measured as the mass remaining upon drying. Approximately 5 g of the plant-based protein hydrogel slurry was pipetted into a small polypropylene dish and the mass was accurately recorded. The dish was placed in a 40° C., oven overnight to dry. The dry mass was measured immediately after removing from the oven and the solids content of the protein hydrogel was calculated as a percentage of the initial wet mass.

### Example 1: Preparation of a Pea Protein Hydrogel Slurry

### a) Protein Hydrogel Formation

450 g of a mixture was prepared consisting of 12.5% (w/w) Pea Protein Isolate in 40% (w/w) lactic acid solution.

The mixture was then heated in a water bath at 80° C., for 30 minutes, followed by a short sonication step to disrupt large colloidal aggregates (Bandelin HD4200 (200 W. 20 kHz, probe TS113, 80% amplitude)), after which a transparent solution was obtained. The energy applied was 100 kJ.

The solution was then poured into a 220 mm petri dish and left to cool down at 5° C., overnight to obtain a self-standing protein hydrogel.

### b) Application of Shear to Protein Hydrogel

Shear was then applied to the hydrogel as follows. The protein hydrogel was cut into ˜1 cm cubes via a low-shear cutting step. The cubes were placed inside a 75 μm filter bag, which was then submerged inside a bucket containing 5 L of deionised water. This formed a coarse protein hydrogel slurry within the filter bag. The hydrogel cubes were left to soak for 1.5 h, with occasional gentle agitation. This step was performed to reduce to concentration of lactic acid in the hydrogel by diffusion to the continuous aqueous phase. It was repeated five more times until the final pH of the aqueous solution was between 3.3 and 3.5.

The strained gel cubes (approx. 360 g) were transferred into a 500 ml bottle with 0.1 wt % sodium benzoate and were exposed to probe sonication in a high shear step (Bandelin HD4200 (200 W. 20 kHz, probe TS113, 40% amplitude): the energy applied was 0.1 kJ per gram of strained gel cubes), so as to form a homogeneous low-viscosity dispersion of fine fragments of proteins.

Steps a and b were carried out in duplicate to produce two samples of slurry which were combined into a single sample and strained through a 75 μm sieve. The pH of the combined and filtered samples was 3.52. The viscosity of the slurry was 16.3 cps at 64 s⁻¹. The solids content was measured as 4.84 wt %.

The prepared slurry was split into three portions.

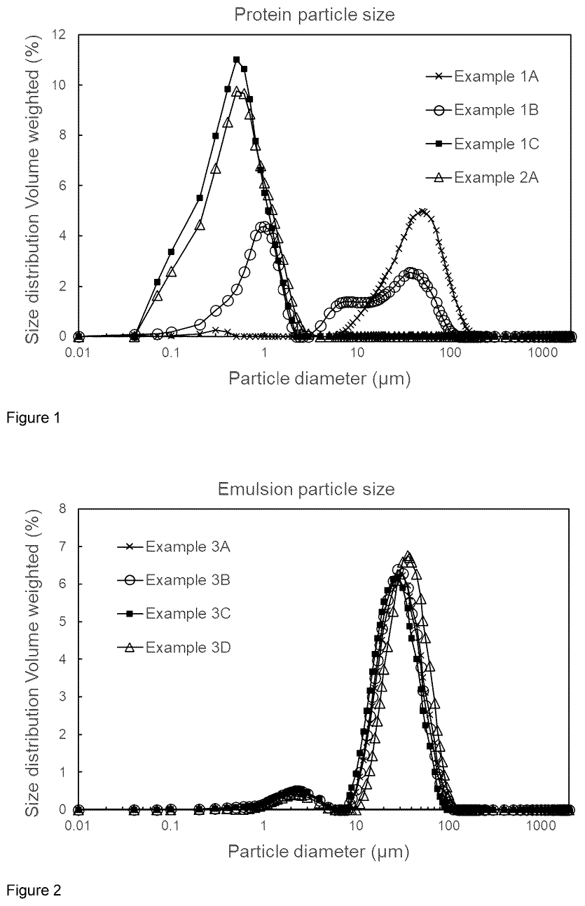

The first portion (Example 1A) was adjusted to pH 3.0 with 85% lactic acid. The particle size of the dispersion was measured and the full distribution is given in FIG. 1. Example 1A had a de of 40.1 μm and a d₉₀ of 85.2 μm.

The second portion (a 100 ml sample, Example 1B), was adjusted to pH 3.0 with 85% lactic acid and ultrasonicated to further reduce the particle size distribution (0.18 kJ/ml ultrasonication). The particle size of the dispersion was measured and the full distribution is given in FIG. 1. Example 1B had a de of 1.30 μm and a de of 38.4 μm.

The third portion (a 100 ml sample. Example 1C), was adjusted to pH 3.0 with 85% lactic acid and ultrasonicated to further reduce the particle size distribution (3.44 kJ/ml ultrasonication). The particle size of the dispersion was measured and the full distribution is given in FIG. 1. Example 1C had a de of 0.36 μm and a de of 0.94 μm

### Example 2: Preparation of Pea Protein Mixture

Example 2A was prepared by suspending of 5.03 g of PPI in 94.6 g of deionized (DI) water, along with 0.1 g of sodium benzoate. The pH was adjusted to 3.0 with 1 M HCl and the sample was sonicated to achieve a small particle size (d₅₀ of less than 0.5 μm), comparable to that in Example 1C. The sonication energy was typically ˜1.5 kJ/g. The particle size of the mixture was measured and the full distribution is given in FIG. 1. Example 2A had a de, of 0.41 μm and a des of 1.10 μm. The viscosity of the mixture was 3.4 cps at 64 s⁻¹.

### Example 3: Preparation of Emulsions and Stability Testing

Four different emulsions were prepared having a 0.1% protein solids content using the protein slurries of Examples 1A-C and the protein mixture of Example 2A according to the formulation given in Table 1 below.

TABLE 1

| Phase | Material | wt % |
|---|---|---|
| Oil | Miglyol ® 812N | 30.00 |
| Aqueous | DI water | q.s. |
| | Slurry of Example 1A, slurry of Example | To give 0.1% |
| | 1B, slurry of Example 1C or mixture of | solids |
| | Example 2A | |

The pH of each aqueous phase was re-adjusted to pH 3.0 if necessary, with 85% lactic acid (for the slurries of Examples 1A, 1B and 1C), and 1 M HCl (for the mixture of Example 2A). For each emulsion a 200 ml batch was prepared by pouring the oil phase into the aqueous phase and homogenizing the resultant mixture with a high shear mixer (Ultraturrax) at 20,000 rpm for 1 minute.

Each of the four different emulsions was split into two portions. The first portion were adjusted to pH 7 using 10 wt % aqueous KOH under constant stirring using a magnetic stirrer. A summary of the four emulsions is given in Table 2 below. The full particle size distribution of the emulsion droplets is very similar for all four emulsions and is given in FIG. 2.

TABLE 2

| | | | | Example 3D |
|---|---|---|---|---|
| | Example 3A | Example 3B | Example 3C | (comparative) |
| Protein from | Slurry of | Slurry of | Slurry of | Mixture of |
| | Example 1A | Example 1B | Example 1C | Example 2A |
| Protein particle | 40.1 | 1.30 | 0.36 | 0.41 |
| size distribution | | | | |
| d50 (μm) | | | | |
| Protein particle | 85.2 | 38.4 | 0.94 | 1.10 |
| size distribution | | | | |
| d90 (μm) | | | | |
| pH adjustment | adjusted to 3 | adjusted to 3 | adjusted to 3 | adjusted to 3 |
| step before | using lactic | using lactic acid | using lactic | using HCl |
| emulsification | acid | | acid | |
| pH measured | 2.99 | 2.99 | 3.01 | 2.99 |
| before | | | | |
| emulsification | | | | |
| pH adjustment | Adjusted to 7 | Adjusted to 7 | Adjusted to 7 | Adjusted to 7 |
| step after | with KOH | with KOH | with KOH | with KOH |
| emulsification | | | | |
| pH measured | 6.91 | 6.94 | 7.01 | 7.07 |
| after | | | | |
| emulsification | | | | |
| Emulsion | 27.4 | 26.3 | 24.0 | 32.8 |
| droplet size | | | | |
| distribution d50 | | | | |
| (μm) | | | | |
| Separation | 0.13 | 0.13 | 0.11 | 1.50 |
| Index: oil | | | | |
| volume/ | | | | |
| emulsion | | | | |
| volume after | | | | |
| centrifugation | | | | |

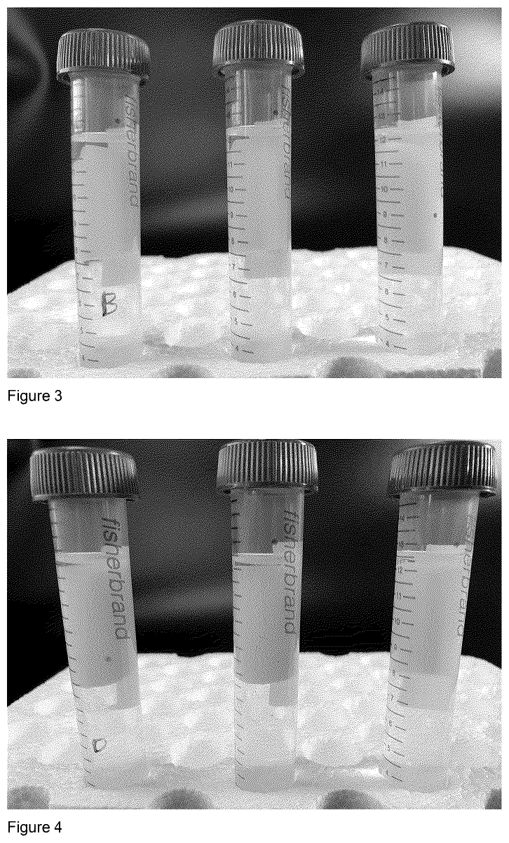

Each emulsion was left at room temperature for between 24 and 72 hours, split between 3 centrifuge tubes (12 g each), then centrifuged at 4347 g for 1 hour 47 minutes to separate out any oil that is not stabilised by the protein. This test therefore gave an indication of the stability of the emulsions, under these stressed conditions. The results are shown in FIGS. 3-6. The Separation Index was calculated as the oil volume of the top layer divided by the volume of the emulsion and is given in Table 2.

FIG. 6 shows that Example 3D separated clearly into a bottom aqueous layer, a middle protein-based emulsion layer and a large top oil layer and the Separation Index was 1.5. This demonstrates that the untreated PPI protein is not able to maintain a stable emulsion of the oil, indicating that long term stability in a product application cannot be expected.

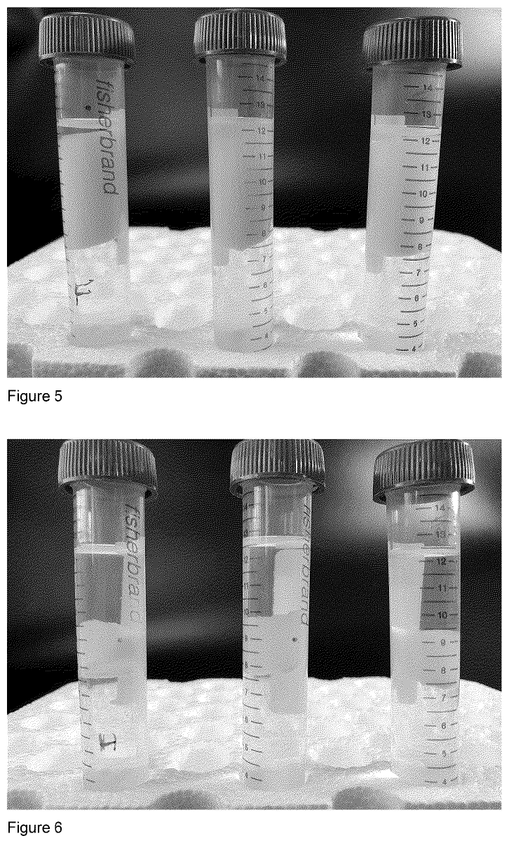

FIG. 5 shows that Example 3C formed a bottom aqueous layer, a middle protein-based emulsion layer and only a very small top oil layer and the Separation Index was 0.11. This is clearly an improvement in terms of emulsion stability over Example 3D made with untreated PPI. Without wishing to be bound by theory, it is thought that the protein aggregates in the protein hydrogel slurries of the present invention have an elongated fine-stranded morphology, which allows them to form a more coherent structural layer around the oil droplets to result in a more stable emulsion even when the particle size distribution is very similar.

FIGS. 3 and 4 show that Example 3A and 3B each formed a bottom aqueous layer and a top protein-based emulsion layer, wherein the oil remained largely emulsified by the protein hydrogel (as only a very small top oil layer was observed). The Separation Indices were both 0.13. This demonstrates that the protein hydrogel slurries having a larger particle size than in Example 3C are still able to keep most of the oil emulsified under these stressed conditions, indicating long term stability in a product application can be expected.

Examples 3A, 3B and 3C demonstrate that a low level of acid treated protein hydrogel with a small particle size is able to emulsify a large level of oil in a near neutral aqueous formulation. The emulsions of the present invention are therefore expected to have a useful application in a number of different product formulations, e.g. cosmetic formulations.

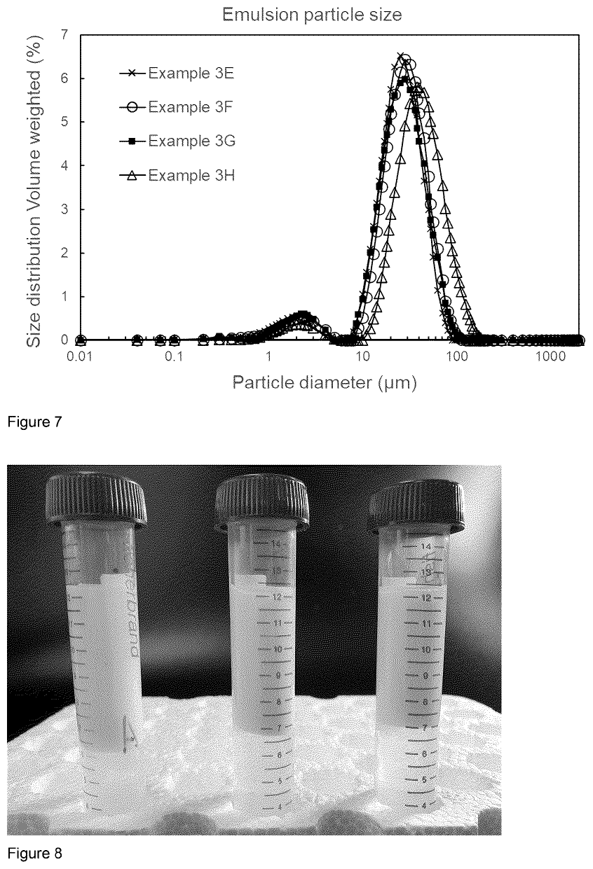

The second portions of the emulsions prepared using the protein slurries of Examples 1A, 1B and 1C and the protein mixture of Example 2A were left without pH adjustment. A summary of the four emulsions is given in Table 3 below. The full particle size distribution of the emulsion droplets is very similar for all four emulsions and is given in FIG. 7.

TABLE 3

| | Example 3E | Example 3F | Example 3G | Example 3H |
|---|---|---|---|---|
| Protein from | Slurry of | Slurry of | Slurry of | Mixture of |
| | Example 1A | Example 1B | Example 1C | Example 2A |
| Protein particle | 40.1 | 1.30 | 0.36 | 0.41 |
| size distribution | | | | |
| d50 (μm) | | | | |
| Protein particle | 85.2 | 38.4 | 0.94 | 1.10 |
| size distribution | | | | |
| d90 (μm) | | | | |
| pH adjustment | adjusted to 3 | adjusted to 3 | adjusted to 3 | adjusted to 3 |
| step before | using lactic | using lactic | using lactic | using HCl |
| emulsification | acid | acid | acid | |
| pH measured | 2.99 | 2.99 | 3.01 | 2.99 |
| before | | | | |
| emulsification | | | | |
| pH adjustment | No pH | No pH | No pH | No pH |
| step after | adjustment | adjustment | adjustment | adjustment |
| emulsification | | | | |
| pH measured | 3.00 | 2.91 | 2.92 | 2.88 |
| after | | | | |
| emulsification | | | | |
| Emulsion | 25.5 | 26.2 | 24.4 | 37.1 |
| droplet size | | | | |
| distribution d50 | | | | |
| (μm) | | | | |
| Separation | 0 | 0 | 0 | 0.03 |
| Index: oil | | | | |
| volume/ | | | | |
| emulsion | | | | |
| volume after | | | | |
| centrifugation | | | | |

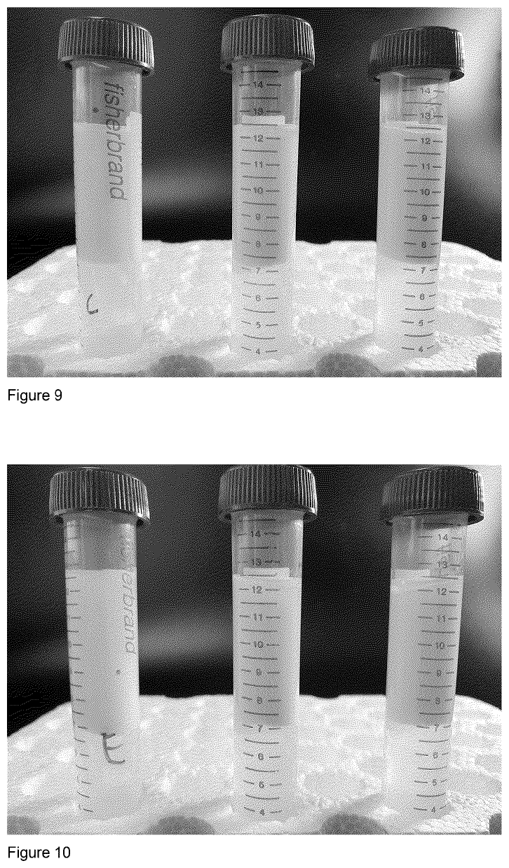

Each emulsion was left at room temperature for between 24 and 72 hours, split between three centrifuge tubes (12 g each), then centrifuged at 4347 g for 1 hour 47 minutes to separate out any oil that is not stabilised by the protein. This test therefore gave an indication of the stability of the emulsions, under these stressed conditions. The results are shown in FIGS. 8-10. The Separation Index was calculated as the oil volume of the top layer divided by the volume of the emulsion and is also given in Table 3.

FIGS. 8-10 show that Examples 3E, 3F and 3G all formed a bottom aqueous layer and a top protein-based emulsion layer, wherein the oil remained fully emulsified by the protein hydrogel. The Separation Indices were therefore zero in all three cases. This demonstrates that the plant-based protein hydrogel slurries of the present invention, at three different particle size distributions, are able to keep the oil emulsified under these stressed conditions, indicating long term stability in a product application can be expected.

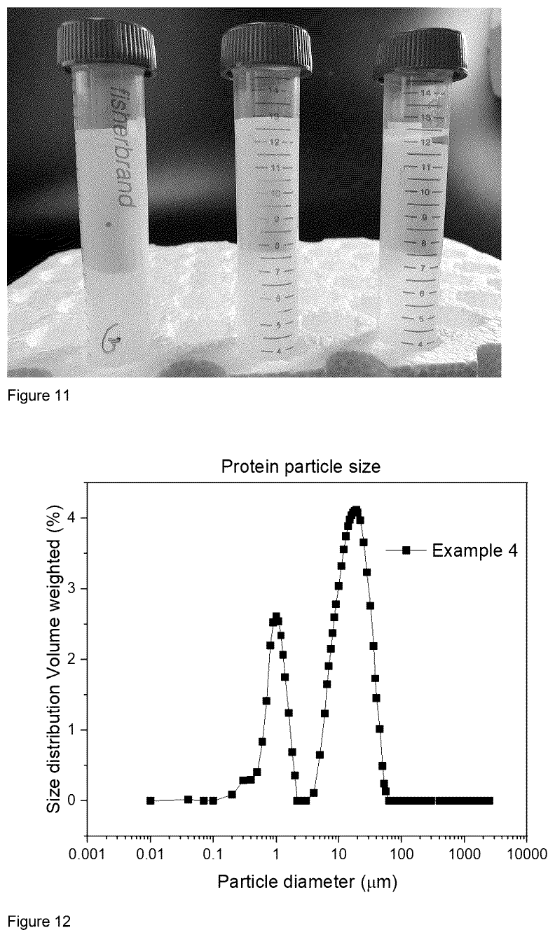

FIG. 11 shows that Example 3H formed a bottom aqueous layer a middle protein-based emulsion layer wherein most of the oil remained emulsified by the protein hydrogel, and a small top oil layer. The Separation Index was calculated as 0.03. This demonstrates that the untreated PPI protein is not able to maintain a fully stable emulsion of the oil, indicating that long term stability in a product application will be difficult for products having an acidic environment.

Without wishing to be bound by theory, it is thought that at acidic pH the amorphous structure of the untreated PPI is able to stabilise the oil droplets by aggregating at the oil droplet interface. This is, however, a less effective stabilisation effect than that of the elongated fine-stranded form of the acid-treated protein hydrogel slurries of the present invention, which form a more coherent structural layer around the oil droplets. Thus, whilst the untreated PPI is able to form an emulsion, it is not as stable as that formed by the acid-treated protein hydrogel slurries. When the pH is increased through the isoelectric point of the pea protein, the untreated PPI aggregates form a non-uniform layer that it is no longer able to stabilise the oil droplet interface. However, under similar conditions, the acid-treated protein retains its morphology to maintain a stable layer at the oil droplet interface. As a result, it is expected that in industrial scale manufacturing processes, where the pH of the product can vary during processing, the acid-treated protein emulsions of the present invention will be stable throughout, whereas emulsions involving untreated PPI would not.

### Example 4: Preparation of a Pea Protein Hydrogel Slurry with Acetic Acid

### a) Preparation of the Protein Hydrogel

1120 g of reverse osmosis (RO) water was added to a 2-litre stainless steel container, and 216 g of pea protein isolate was added. The container was placed in a 92° C. water bath and mixed with an overhead stirrer at 1500 rpm. After stirring for 3 minutes, 480 g of glacial acetic acid was added. The mixture was stirred for 15 minutes at 1500 rpm, then for 30 minutes at 1200 rpm, ensuring that the temperature of the mix was above 85° C., for at least 10 minutes. The mixture was poured into trays to a depth of approximately 10 mm, and left at room temperature overnight.

### b) Application of Shear to Protein Hydrogel

Shear was then applied to the hydrogel as follows. The protein hydrogel was cut into ˜1 cm cubes via a low-shear cutting step. The cubes were split between two 75 micron filter bags, which were each then submerged inside a bucket containing 16 L of RO water. This formed a coarse protein hydrogel slurry within the filter bag. The hydrogel cubes were left to soak, with agitation from an overhead stirrer at 600-800 rpm, for 90-150 min. This step was performed to reduce the concentration of acetic acid in the hydrogel by diffusion to the continuous aqueous phase. The pH of the wash water was then measured, and if it was above 3.2, soaking was continued for a further 30 minutes. If it was below 2.9, half of the water was drained and replaced with fresh RO water, then soaking was continued for a further 30 minutes. The filter bag was then suspended above the bucket to drain for 5 minutes. The washed gel from both filter bags was transferred to a 5 liter beaker, and homogenized with a Silverson mixer at 5000 rpm for 5 minutes. 6000 rpm for 5 minutes, and 7000 rpm for 5 minutes. The smooth slurry was then transferred to 1 L Nalgene bottles (800 g in each), and exposed to high shear ultrasonication (Hielscher UP500Hdt) with cooling on ice, until 250 kJ had been applied, with shaking every 75 kJ. The hydrogel slurry was then passed through a 200 micron sieve before use.

The pH of the filtered sample was 2.9. The protein solids content was measured according to the method herein enclosed as 9.5% wt. The particle size of the slurry was measured according to the method herein enclosed and the full particle size distribution of the dispersion is given in FIG. 12. The dec of the sample was reported as 11.0 μm, and the doo as 28.8 μm.

### Example 5: Preparation of Emulsions and Stability Testing with a Fragrance and Thymol

The Fragrance A was prepared with the fragrance materials in Table 4 and then mixed with Miglyo® I 812N in an 80:20 weight ratio.

TABLE 4

| | | Vapour | | | | | | Fragrance |
|---|---|---|---|---|---|---|---|---|
| Fragrance | CAS | pressure | Log | HSP | | | | A |
| material | number | (Torr) | P | δD | δP | δH | Functionality | (wt %) |
| Geraniol | 106-24-1 | 0.013 | 3.42 | 16.9 | 4.2 | 7.6 | Primary | 25 |
| | | | | | | | alcohol | |
| Undecavertol | 81782-77-6 | 0.011 | 3.97 | 16.4 | 3.4 | 7.5 | Secondary | 25 |
| | | | | | | | alcohol | |
| Delta | 57378-68-4 | 0.010 | 3.56 | 16.9 | 3.5 | 2.6 | Ketone | 25 |
| damascone | | | | | | | | |
| dodecane | 2437-25-4 | 0.005 | 5.12 | 16.0 | 5.4 | 3.1 | Nitrile | 25 |
| nitrile | | | | | | | | |

Two different emulsions were prepared having a 0.9% protein solids content using the protein slurry of Example 4 according to the formulations given in Table 5 below.

TABLE 5

| | | Example | Example |
|---|---|---|---|
| Phase | Material | 5A | 5B |
| Oil | Solution of 80% Fragrance A in | 30.0 wt % | 0 |
| | Miglyol ® 812N | | |
| | Solution of 40% Thymol in | 0 | 30.0 |
| | Miglyol ® 812N | | |
| Aqueous | DI water | q.s. | q.s. |

| | |
|---|---|
| Slurry of Example 4 | To give 0.9 wt % |
| | protein solids |

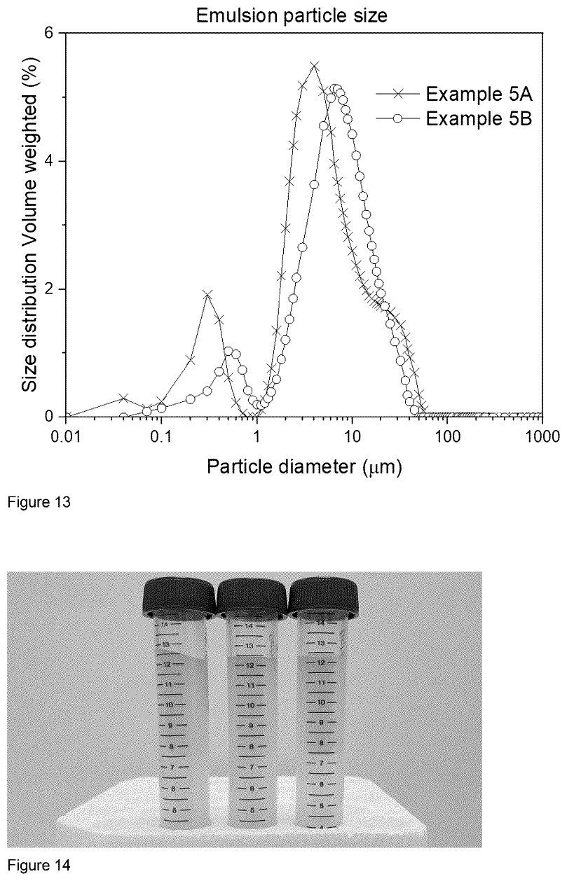

For each emulsion a 200 ml batch was prepared by pouring the oil phase into the aqueous phase and homogenizing the resultant mixture with a high shear mixer Silverson LSM-A at 10000 rpm for 5 minutes. The full particle size distribution of the emulsion droplets is very similar for both emulsions and is given in FIG. 13.

TABLE 6

| | Example 5A | Example 5B |
|---|---|---|
| Protein from | Slurry of Example 4 | Slurry of Example 4 |
| Protein particle size distribution d50 (μm) | 11.0 | 11.0 |
| Protein particle size distribution d90 (μm) | 28.8 | 28.8 |
| pH adjustment step before emulsification | No pH adjustment | No pH adjustment |
| pH measured before emulsification | 2.91 | 2.91 |
| pH adjustment step after emulsification | No pH adjustment | No pH adjustment |
| pH measured after emulsification | 2.88 | 2.91 |
| Emulsion droplet size distribution d50 (μm) | 3.7 | 5.9 |
| Emulsion droplet size distribution d90 (μm) | 18.1 | 16.8 |
| Separation Index: oil volume/emulsion | 0 | 0 |
| volume after centrifugation | | |

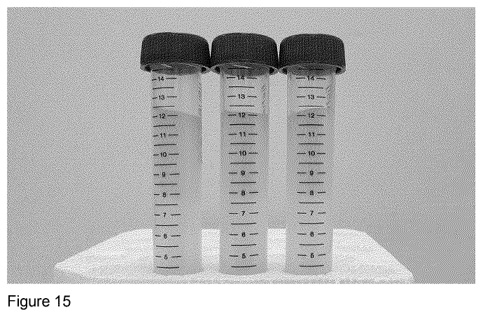

Each emulsion was left at room temperature for between 24 and 72 hours, split between 3 centrifuge tubes (12 g each), then centrifuged at 4347 g for 1 hour 47 minutes to separate out any oil that is not stabilised by the protein. This test therefore gave an indication of the stability of the emulsions, under these stressed conditions. The results are shown in FIGS. 14 and 15 for Examples 5A and 5B respectively, where both examples formed a bottom aqueous layer and a top protein-based emulsion layer, wherein the oil remained fully emulsified by the protein hydrogel. The Separation Index was calculated as the oil volume of the top layer divided by the volume of the emulsion and is given in Table 6. For both Examples 5A and 5B no separate oil phase formed so the Separation Index was zero for both, demonstrating that the plant-based protein hydrogel slurries of the present invention are able to keep a variety of oils emulsified under these stressed conditions, indicating long term stability in a product application can be expected.

### Example 6: Coating of Fragrance Emulsions on Card

The emulsion Example 5A was further diluted to 1 wt % of Fragrance A and coated onto 210 gsm white card. A piece of card approximately 14×21 cm in size was clipped to a rigid aluminium sheet, and 20 ml of the diluted emulsion was applied. A 300 μm spiral bar coater (Elcometer) was used to coat the card, and the excess emulsion was removed. The card was left to dry in a fume cupboard overnight, and cut into 5×9 cm pieces for olfactive evaluation by an expert panel according to the following scale:

| Odour strength | Description |
|---|---|
| 5 | Very Strong |
| 4 | Strong |
| 3 | Moderate |
| 2 | Weak |
| 1 | Very Weak |
| 0 | No odour |

The average grade of 3 indicated a moderate fragrance strength with a fruity, sweet, orange peel character.

1. A method for the preparation of an emulsion, the method comprising:
(a) forming a solution comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s);
(b) inducing the protein in the solution to undergo a sol-gel transition to form a plant-based protein hydrogel;
(c) subjecting the plant-based protein hydrogel to a first shear step to form a first plant-based protein hydrogel slurry;
(d) subjecting the first plant-based protein hydrogel slurry to a solvent reduction step to form a washed plant-based protein hydrogel slurry;
(e) subjecting the washed plant-based protein hydrogel slurry to a second shear step to form a second plant-based protein hydrogel slurry; and
(f) dispersing a lipophilic phase in the second plant-based protein hydrogel slurry to form the emulsion,
wherein said emulsion has a protein solids content of less than 1 wt % based upon the total weight of the emulsion, and wherein said second plant-based protein hydrogel slurry has a doo of less than 250 μm as measured by laser diffraction.

2. The method according to claim 1, wherein the plant-based protein(s) is selected from the group consisting of pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein.

3. The method according to claim 1, wherein the first co-solvent is an organic acid selected from the group consisting of, acetic acid, lactic acid, formic acid, gluconic acid, propionic acid, an α-hydroxy acid, and/or a p-hydroxy acid.

4. The method according to claim 1, wherein the second co-solvent is selected from water, ethanol, and/or ethyl acetate.

5. The method according to claim 1, wherein in step (b) the protein solution is heated to a first temperature above the sol-gel transition temperature of the one or more plant-based protein(s) solution, then reduced to a second temperature below the sol-gel transition temperature of the one or more plant-based protein(s) solution to form a hydrogel.

6. A method according to claim 1, wherein said first shear step involves fragmenting the plant-based protein hydrogel into fragments, wherein said fragments produced in said first shear step have a particle dimension in the range 1 mm to 100 mm as determined by optical microscopy.

7. A method according to claim 1, wherein said solvent reduction step comprises the steps of:
(i) contacting the first plant-based protein hydrogel slurry with a non-solubilising solvent;
(ii) separating the first plant-based hydrogel slurry from the non-solubilising solvent to give a washed plant-based protein hydrogel slurry; and
(iii) optionally repeating steps (i) and (ii).

8. A method according to claim 1, wherein said second shear step involves further fragmenting the plant-based protein hydrogel wherein said second plant-based protein hydrogel slurry has a d₅₀ of less than 100 μm as measured by laser diffraction.

9. A method according to claim 1, wherein said second plant-based protein hydrogel slurry is subjected to a pH adjustment step between steps (e) and (f).

10. A method according to claim 1, wherein said lipophilic phase comprises a solvent, a butter, or a wax.

11. A method according to claim 10, wherein said lipophilic phase comprises a solvent selected from a fatty acid ester, a fatty acid, a linear or branched hydrocarbon of natural mineral or synthetic origin, a fatty alcohol or ether thereof, a vegetable oil, a silicone oil, a phthalate ester, a rosin resin, a diol, a triol, benzyl benzoate, triethyl citrate and triacetin, or combinations thereof.

12. A method according to claim 1, wherein said lipophilic phase comprises an active ingredient(s), which is preferably selected from the group consisting of a vitamin, a mineral, a flavour material, a fragrance material, a pro-flavour, a pro-fragrance, a flavour enhancer, a malodour counteractant, a nutraceutical, a probiotic, a pharmaceutical, an anti-microbial agent, an anti-viral agent, an anti-inflammatory agent, an antioxidant, a pesticide, a herbicide, a fertiliser, a fungicide, an insecticide, an animal repellent, an antiacne agent, an anti-ageing agent, a skin lightening agent, an emollient, a humectant, an occlusive agent, a skin moisturizing agent, an antiperspirant or deodorant agent, a wrinkle control agent, a fabric softener active, a surface cleaning active, a skin conditioning agent, a hair conditioning agent, a sunscreen, a dye, a pigment, and an adhesive, and combinations thereof.

13. A method according to claim 12, wherein said active ingredient(s) is at least one fragrance material or flavour material and wherein:
the at least one fragrance material or flavour material has a vapour pressure greater than or equal to 0.0001 Torr at 25° C.; and/or
the at least one fragrance material or flavour material has a log P greater than or equal to 3.0.

14. A method according to claim 12, wherein said active ingredient(s) is at least one fragrance material or flavour material which is part of a fragrance or flavour, wherein the fragrance or flavour contains at least 20 wt % of fragrance material(s) or flavour material(s) with a log P greater than or equal to 3.0 based upon the total weight of the fragrance or flavour.

15. A method according to claim 12, wherein said active ingredient(s) is a vitamin or a mineral, selected from the group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, magnesium, sodium, potassium, zinc, iron, calcium, iodine, omega 3, folic acid, thiamin, riboflavin, niacin and phosphorous, and mixtures thereof.

16. (canceled)

17. A method according to claim 1, wherein the emulsion has a protein solids content of less than 0.9 wt % based upon the total weight of the emulsion.

18. A method according to claim 1, further comprising altering the pH of the emulsion such that it is different to the isoelectric point of the plant-based protein by more than 1 pH unit and is in the range of 5.5 to 7.5.

19. (canceled)

20. (canceled)

21. An emulsion obtained by or obtainable by the method of claim 1.

22. An emulsion comprising a lipophilic phase dispersed in a plant-based protein hydrogel slurry comprising a plant-based protein(s), wherein said emulsion has a protein solids content of less than 1 wt % based upon the total weight of the emulsion.

23. An emulsion according to claim 22, wherein the emulsion has a protein solids content of less than 0.9 wt % based upon the total weight of the emulsion.

24. An emulsion according to claim 22, wherein the plant-based protein(s) is selected from the group consisting of pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein.

25. An emulsion according to claim 22, wherein the plant-based protein(s) has been pre-treated with an organic acid selected from the group consisting of acetic acid, lactic acid, formic acid, gluconic acid, propionic acid, an a-hydroxy acid, and/or a p-hydroxy acid.

26. An emulsion according to claim 22, wherein;
the plant-based protein hydrogel slurry has a d₅₀ of less than 100 μm as measured by laser diffraction; and/or
the plant-based protein hydrogel slurry has a do of less than 250 μm as measured by laser diffraction.

27. An emulsion according to claim 22, wherein said lipophilic phase comprises a solvent, a butter or a wax.

28. An emulsion according to claim 27, wherein said lipophilic phase comprises a solvent selected from a fatty acid ester, a fatty acid, a linear or branched hydrocarbon of natural mineral or synthetic origin, a fatty alcohol or ether thereof, a vegetable oil, a silicone oil, a phthalate ester, a rosin resin, a diol, a triol, benzyl benzoate, triethyl citrate and triacetin, or combinations thereof.

29. An emulsion according to claim 22, wherein said lipophilic phase comprises an active ingredient(s), preferably selected from the group consisting of a vitamin, a mineral, a flavour material, a fragrance material, a pro-flavour, a pro-fragrance, a flavour enhancer, a malodour counteractant, a nutraceutical, a probiotic, a pharmaceutical, an anti-microbial agent, an anti-viral agent, an anti-inflammatory agent, an antioxidant, a pesticide, a herbicide, a fertiliser, a fungicide, an insecticide, an animal repellent, an antiacne agent, an anti-ageing agent, a skin lightening agent, an emollient, a humectant, an occlusive agent, a skin moisturizing agent, an antiperspirant or deodorant agent a wrinkle control agent, a fabric softener active, a surface cleaning active, a skin conditioning agent, a hair conditioning agent, a sunscreen, a dye, a pigment, and an adhesive, and combinations thereof.

30. An emulsion according to claim 29, wherein said active ingredient(s) is at least one fragrance material or flavour material and wherein:
the at least one fragrance material or flavour material has a vapour pressure greater than or equal to 0.0001 Torr at 25° C.; and/or
the at least one fragrance material or flavour material has a log P greater than or equal to 3.0.

31. An emulsion according to claim 29, wherein said active ingredient(s) is at least one fragrance material or flavour material which is part of a fragrance or flavour, wherein the fragrance or flavour contains at least 20 wt % of fragrance material(s) or flavour material(s) with a log P greater than or equal to 3.0 based upon the total weight of the fragrance or flavour.

32. An emulsion according to claim 29, wherein said active ingredient(s) is a vitamin or a mineral, selected from the group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, magnesium, sodium, potassium, zinc, iron, calcium, iodine, omega 3, folic acid, thiamin, riboflavin, niacin and phosphorous, and mixtures thereof.

33. An emulsion according to claim 22, wherein the pH of the emulsion is such that it is different to the isoelectric point of the plant-based protein by more than 1 pH unit.

34. An emulsion according to claim 22, wherein the pH of the emulsion is in the range 5.5 to 7.5.

35. An emulsion according to claim 22, wherein the plant-based protein(s) have a protein secondary structure with at least 40% intermolecular β-sheet, wherein the % intermolecular β-sheet content is measured by FTIR.

36. A composition comprising an emulsion prepared according to the method of claim 1.

37. A composition according to claim 36 which is a cosmetic composition, a fragrance composition (e.g. a fine fragrance or a laundry scent booster), a beverage composition, an oral care composition, a pharmaceutical composition, or an agricultural composition.

38. The use of a plant-based protein hydrogel slurry as an emulsifier in a composition.

39. The use according to claim 38, wherein the composition is a cosmetic composition, a fragrance composition (e.g. a fine fragrance or a laundry scent booster), a beverage composition, an oral care composition, a pharmaceutical composition, or an agricultural composition.
